(19) United States
(12) Patent Application Publication
(10) Pub. No.: US 2021/0292744 A1
(43) Pub. Date: Sep. 23, 2021

(54) EXTRACTION OF POLYNUCLEOTIDES

(71) Applicant: Monsanto Technology LLC, St. Louis, MO (US)

(72) Inventors: Gregory James Bunkers, Wildwood, MO (US); Megan E. Gillespie, St. Louis, MO (US); Dennis H. Yang, Ballwin, MO (US)

(73) Assignee: Monsanto Technology LLC, St. Louis, MO (US)

(21) Appl. No.: 17/258,414

(22) PCT Filed: Jul. 11, 2019
(86) PCT No.: PCT/US2019/041368

§ 371 Date: Jan. 6, 2021

Related U.S. Application Data
(60) Provisional application No. 62/696,371, filed on Jul. 11, 2018.

(51) Int. Cl. C12N 15/10; G01N 33/24

(52) U.S. Cl. CPC C12N 15/1003; G01N 33/24

(57) ABSTRACT

The present invention relates to various methods and apparatus for extracting polynucleotides, particularly in soil samples. Further methods and apparatus of the present invention provide for additional processing of the polynucleotides, such as analysis and/or amplification. Still other methods and apparatus relate to applying a treatment to a plant or seed based on the analysis of the polynucleotide.

### FIELD OF THE INVENTION

The present invention relates to various methods and apparatus for extracting polynucleotides, particularly in soil samples. Further methods and apparatus of the present invention provide for additional processing of the polynucleotides, such as analysis and/or amplification. Still other methods and apparatus relate to treating a plant or plant part against a pathogen based on the analysis of the polynucleotide.

### BACKGROUND OF THE INVENTION

The microbiome of a plant is particularly important for the health and survival of the plant. Microorganisms in the rhizosphere (area near the roots) can supply beneficial nutrients and resources to a growing plant, but pathogens can reduce plant health, survival, productivity, or yield. Thus, to protect plants and/or enhance plant survival, productivity, or yield, it is necessary to accurately detect and quantify these organisms and apply targeted treatments if needed.

Pathogens and other organisms (e.g., microorganisms and plants) can be detected by isolating and extracting polynucleotides (e.g., DNA, RNA and other nucleic acids) from soil. However, soil is a complex matrix containing a wide variety of components. Some of the components in soil can inhibit the extraction and/or analysis of the target polynucleotide. Also, there are many different types of soil and soil conditions. Consequently, extracting polynucleotides from soil in an efficient, selective, and consistent manner is challenging. Therefore, a need exists for effective methods and apparatus for extracting polynucleotides from soil. There is a further need for high throughput methods and apparatus capable of efficiently extracting polynucleotides from a large sample collection.

### BRIEF SUMMARY OF THE INVENTION

In various aspects, the present invention is directed to methods for preparing an extract comprising polynucleotides comprising mixing a sample comprising particulates and the polynucleotides with (1) a macroporous compound component comprising a macroporous compound, (2) a base, and (3) a solvent to form an extraction mixture, wherein the sample is obtained from a growing area; and separating at least a portion of the polynucleotides from the extraction mixture to form the extract comprising polynucleotides and a first fraction comprising at least a portion of the macroporous compound component and particulates, wherein the concentration of particulates in the extraction mixture is greater than the concentration of the particulates in the extract.

In other aspects, the present invention is directed to methods for analyzing a polynucleotide comprising preparing an extract comprising polynucleotides according to any of the methods described herein; and detecting or identifying the polynucleotide in the extract. Still further aspects are directed to methods for detecting a pathogen in a sample comprising soil particulate comprising preparing an extract comprising polynucleotides from the sample according to any of the methods described herein, analyzing a polynucleotide obtained from the extract, and detecting the pathogen in the sample based on the analysis of the polynucleotide.

Further aspects of the present invention are directed to methods for treating a plant or plant part thereof comprising preparing an extract comprising polynucleotides according to any of the methods described herein, analyzing a polynucleotide obtained from the extract and applying to the plant, plant part, or locus thereof (i.e., a location sufficiently proximate to the plant or plant part for effective treatment) a treatment based on the analysis of the polynucleotide.

Additional aspects of the present invention are directed to a mobile soil analysis system comprising: a soil sample collection device; at least one vessel sized and shaped to receive the soil sample and one or more analysis reagents; a polynucleotide detector configured to receive at least a portion of the soil sample and one or more analysis reagents from the at least one vessel and identify and/or quantify polynucleotides in the soil sample, wherein the polynucleotide detector is configured to generate a polynucleotide signal; a soil sample processor in communication with the polynucleotide detector and configured to analyze the soil sample at least in part based on the polynucleotide signal; a tangible storage medium storing soil sample analysis instructions executable by the soil sample processor, wherein when the soil sample analysis instructions are executed by the soil sample processor, the polynucleotide signal is processed and the analytic data associated with the soil sample is stored on the tangible storage medium; and a mobile platform supporting the at least one vessel and the polynucleotide detector.

Further aspects of the present invention are directed to a mobile soil treatment system comprising: the mobile soil analysis system described herein; a container for receiving an agrochemical formulation; and a dispenser for administering the agrochemical formulation to a soil collection, a growing area, a plant, a plant part, and/or locus thereof, wherein the dispenser is in fluid communication with the container.

Other objects and features will be in part apparent and in part pointed out hereinafter.

### DETAILED DESCRIPTION OF THE INVENTION

Various processes for preparing extracts of polynucleotides from environmental samples are provided. In various aspects, preparation of polynucleotide extracts can comprise mixing an environmental sample comprising polynucleotides with a macroporous compound component, a base, and a solvent and separating at least a portion of the polynucleotides from the extraction mixture to form the extract comprising polynucleotides and a first fraction comprising at least a portion of the macroporous compound component. The polynucleotide extract described herein can be used in any application recognized by one of ordinary skill in the art. Further aspects of the invention are directed to the analysis of the polynucleotide extract. In some aspects, the polynucleotide extract can be analyzed to detect or quantify an organism or agent comprising polynucleotides in the environmental sample. Also provided are high throughput methods and apparatuses designed to perform the various embodiments of the invention efficiently across a large sample size.

In various embodiments, processes for preparing extracts comprising polynucleotides from a sample are provided. For example, some processes for the preparation of a polynucleotide extract can comprise mixing a sample comprising particulates and the polynucleotides with (1) a macroporous compound component comprising a macroporous compound, (2) a base, and (3) a solvent to form an extraction mixture, wherein the sample is obtained from a growing area; and separating at least a portion of the polynucleotides from the extraction mixture to form the extract comprising polynucleotides and a first fraction comprising at least a portion of the macroporous compound component and the particulates, wherein the particulates in the extraction mixture is greater than the concentration of particulates in the extract comprising polynucleotides.

Other aspects of the present invention include processes for analyzing a polynucleotide. In various embodiments, these processes comprise preparing an extract containing polynucleotides and then detecting or identifying a polynucleotide in the extract. In additional embodiments, these processes can further comprise detecting or quantifying an organism or agent comprising polynucleotides in the sample based on the identity or quantity of the polynucleotide in the extract. Further, in various embodiments, methods directed to treatment of seeds or plants against the identified pathogens are described.

Conventional methods for extracting polynucleotides from environmental samples (e.g., from soil or plant material) are hindered by two primary issues that reduce the quality and usefulness of the extract or that reduce the efficiency or convenience of the procedures. First, co-extraction of various inhibitors can interfere with or block downstream processing of the extract, but diluting the sample to avoid inhibition can sometimes reduce the level of polynucleotides under a detection threshold. The present invention provides various methods that reduce the amount of components in the extract that can interfere with polynucleotide analysis without significantly reducing polynucleotide levels, resulting in a sample that can be analyzed more efficiently. Second, current methods for extracting polynucleotides require specialized equipment/reagents and complicated steps in a laboratory to perform reliably. The expense and time of using these methods makes them inefficient for analyzing the numerous samples that can be collected across an entire growing area, such as, for example, when samples are taken using a plant by plant or plot by plot sampling strategy. To address this problem, various embodiments allow for the preparation of the extract, and analyses of the nucleotide sequences within the extract, at the sample site. The methods disclosed herein require relatively inexpensive reagents, and do not require specialized lab equipment, allowing them to be completed quickly. The ability to perform the methods on-site can save time that is typically lost when transferring samples to a laboratory. Therefore, the methods described herein can provide for “on-site” collection, polynucleotide extraction, and analysis of a large number of samples

### Methods for Preparing Extracts of Polynucleotides

As noted, conventional methods for extracting polynucleotides from environmental samples are sometimes hindered by the co-extraction of various components that interfere with downstream processing of the polynucleotide extract. Diluting a sample can reduce inhibition or interference, but can also reduce polynucleotide levels below detection levels. It has been discovered that certain macroporous compounds are effective in reducing levels of inhibitors without reducing the levels of polynucleotides from environmental samples. The methods described herein are particularly suited for preparing extracts of polynucleotides from soil samples or plant material.

Various methods for preparing an extract comprising polynucleotides comprise: mixing a sample comprising particulates and the polynucleotides with (1) a macroporous compound component comprising a macroporous compound, (2) a base, and (3) a solvent to form an extraction mixture and separating at least a portion of the polynucleotides from the extraction mixture to form the extract comprising polynucleotides and a first fraction comprising at least a portion of the macroporous compound component and particulates, wherein the concentration of particulates in the extraction mixture is greater than the concentration of the particulates in the extract. In some embodiments, the concentration of particulates in the first fraction is greater than the concentration of the particulates in the extract.

In various embodiments, the sample comprising particulates and polynucleotides is obtained from a growing area. As used herein, “growing area” is any area or facility where plants are grown. Non-limiting examples include fields, cultivated fields, greenhouses, growth chambers, pots, or any other industrial, academic, public or private setting where multiple plants are grown for study and/or consumption. In some embodiments, the sample can be obtained from the rhizosphere of the plant. In various embodiments, the sample can be obtained from a crop growing area.

As noted, the sample comprises particulates. In some embodiments, the particulates comprise soil particulates and/or one or more plant parts. Soil and/or soil particulates generally include various mediums in which plants grow. Various types of soil include, for example, loam, sand, peat, clay, silty clay loam, loamy sand, clay loam, silt loam, sandy loam, or combinations thereof. Soil and/or soil particulates can be unprocessed or processed (physically or chemically processed). Physical processing of soil includes, for example, compacting the soil and/or reducing the particle size of the soil particulates. In some cases, the sample is a soil core obtained from the growing area. A plant part, as used herein, refers to a whole plant, any part thereof, or a cell or tissue culture derived from a plant, comprising any of: whole plants, plant components or organs (e.g., leaves, stems, roots, etc.,), plant tissues, seeds, plant cells, and/or progeny of the same. For example, a plant part can be selected from the group consisting of a leaf, stem, root, seed, and combinations thereof. A plant cell is a biological cell of a plant, taken from a plant or derived through culture from a cell taken from a plant. In various embodiments, the plant comprises any crop plant. A crop plant can be any plant grown or cultivated for human and/or animal use.

The sample comprising particulates and polynucleotides can also be obtained from soil comprising at least one plant or plant part (e.g., seed). In various embodiments, the sample is obtained from a growing area containing at least one plant or plant part (e.g., seed). In various embodiments, the sample obtained from a growing area has a moisture content of no greater than about 50 wt. %, no greater than about 25 wt. %, no greater than about 10 wt. %, or no greater than about 5 wt. %.

The sample comprises polynucleotides from any source. As used herein, the term “polynucleotide” refers to a nucleic acid molecule containing multiple nucleotides and generally refers both to “oligonucleotides” (a polynucleotide molecule of less than 26 nucleotides in length, e.g., 18-25) and polynucleotides of 26 or more nucleotides. The polynucleotides described herein can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments include those wherein the polynucleotide is selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used. In various embodiments, the polynucleotides can comprise single-stranded DNA (ssDNA), single-stranded RNA (ssRNA), double-stranded DNA (dsDNA), double-stranded RNA (dsRNA), RNA/DNA hybrid and combinations thereof.

The term “dsRNA” refers to a molecule comprising two antiparallel ribonucleotide strands bound together by hydrogen bonds, each strand of which comprises ribonucleotides linked by phosphodiester bonds running in the 5′-3′ direction in one and in the 3′-5′ direction in the other. Two antiparallel strands of a dsRNA can be perfectly complementary to each other or comprise one or more mismatches up to a degree where any one additional mismatch causes the disassociation of the two antiparallel strands. A dsRNA molecule can have perfect complementarity over the entire dsRNA molecule, or comprises only a portion of the entire molecule in a dsRNA configuration. Two antiparallel strands of a dsRNA can also be from a continuous chain of ribonucleotides linked by phosphodiester bonds, e.g., a hairpinlike structure (often also called a stem-loop structure).

The sample comprising particulates and polynucleotides can further comprise at least one inhibitor. An inhibitor can comprise a substance that interferes with downstream analysis or processing of the extract comprising polynucleotides. For example, inhibitors can include proteinases, polysaccharides, heme (blood), urea, heavy metals and humic substances (e.g., humin, humic acids, fulvic acids). In various embodiments, the inhibitor comprises humic acid.

In various embodiments, the sample comprising polynucleotides and particulates can further comprise cellular or viral components that collectively comprise at least a portion of the polynucleotides. These components can comprise whole or partial organisms, whole cells, or cell fragments, viruses or viral particles. Cell fragments can further comprise organelles (e.g., nuclei, ER, mitochondria, vesicles, Golgi, lysosomes). In various methods described herein, the sample is mixed with a macroporous compound component, a base, and a solvent to form an extraction mixture.

The extraction mixture comprises a macroporous compound component. In general, the macroporous compound component comprises a macroporous compound. Macroporous compounds are polymers forming discrete particles having multiple holes (pores) on their surface (e.g., macroporous resins). These pores increase the surface area of the particle, allowing for increased binding and chelation of various molecules. The macroporous compounds useful in the methods described herein can be defined by the polymeric composition, mean pore size and pore volume, specific surface area, density and other parameters. In various embodiments, the macroporous compound comprises a macroporous resin.

In various embodiments, the macroporous compound comprises a polymethacrylic polymer, a polymethylmethacrylic polymer, or any combination thereof. In some embodiments, the macroporous compound comprises DAX-8, XAD-7, XAD-16, and/or hydrates thereof. In certain embodiments, the macroporous compound comprises a hydrated macroporous resin. For example, the macroporous compound can comprise any hydrated form of DAX-8, XAD-7, and/or XAD-16. In some embodiments, the hydrated macroporous resin can have a density of from about 1 to about 1.2 g/mL, from about 1 to about 1.1 g/mL, or from about 1.05 to about 1.1 g/mL at 25° C. In various embodiments, the macroporous compound is DAX-8, which is available from Sigma-Aldrich.

The macroporous compound can have a mean pore diameter from about 5 nm to about 100 nm, from about 5 nm to about 50 nm, from about 5 nm to about 30 nm, from about 10 nm to about 100 nm, from about 10 nm to about 50 nm, from about 10 nm to about 30 nm, from about 20 nm to about 100 nm, from about 20 nm to about 50 nm, from about 20 nm to about 30 nm, or from about 20 nm to about 25 nm (e.g., 22.5 nm). Mean pore diameters are determined in accordance with the procedures described in E. P. Barrett, L. G. Joyner, P. P. Halenda, J. Am. Chem. Soc. 1951, 73, 373-380 (BJH method), which is incorporated herein by reference.

The macroporous compound can have a specific surface area of about 50 m²/g to about 1000 m²/g, from about 50 m²/g to about 800 m²/g, from about 50 m²/g to about 500 m²/g, from about 50 m²/g to about 200 m²/g, from about 100 m²/g to about 1000 m²/g, from about 100 m²/g to about 800 m²/g, from about 100 m²/g to about 500 m²/g, from about 100 m²/g to about 300 m²/g, from about 100 m²/g to about 200 m²/g, or from about 125 m²/g to about 175 m²/g (e.g., about 140 m²/g). The specific surface area can be determined from by Brunauer, Emmett and Teller method. See the methods described in J. Am. Chem. Soc. 1938, 60, 309-331, which is incorporated herein by reference.

It has been discovered that, in some instances, the effectiveness of the extraction procedure can depend on the concentration of macroporous compound component in relation to the other components (e.g., particulates) in the extraction mixture. In some embodiments, the ratio of the mass (g) of the macroporous compound component to the volume (cm³) of the sample comprising particulates is at least about 1:1, at least about 1.2:1, at least about 1.4:1, or at least about 1.6:1. For example, the ratio of the mass (g) of the macroporous compound component to the volume (cm³) of the particulates can be from about 1:1 to about 2:1, from about 1.2:1 to about 2:1, from about 1.4: to about 2:1, from about 1.6:1 to about 2:1, from about 1:1 to about 1.8:1, from about 1.2:1 to about 1.8:1, from about 1.4: to about 1.8:1, from about 1.6:1 to about 1.8:1, from about 1:1 to about 1.6:1, from about 1.2:1 to about 1.6:1, or from about 1.4:1 to about 1.6:1 (e.g., about 1:5:1).

In some embodiments, the ratio of the mass (g) of the macroporous compound component to the mass (g) of the particulates in the extraction mixture is at least about 1:1, at least about 1.2:1, at least about 1.4:1, or at least about 1.6:1. For example, the ratio of the mass (g) of the macroporous compound component to the mass (g) of the particulates can be from about 1:1 to about 2:1, from about 1.2:1 to about 2:1, from about 1.4: to about 2:1, from about 1.6:1 to about 2:1, from about 1:1 to about 1.8:1, from about 1.2:1 to about 1.8:1, from about 1.4: to about 1.8:1, from about 1.6:1 to about 1.8:1, from about 1:1 to about 1.6:1, from about 1.2:1 to about 1.6:1, or from about 1.4:1 to about 1.6:1 (e.g., about 1:5:1).

In various embodiments, the macroporous compound component can also comprise a portion of the solvent (e.g., water) or other diluent. In some embodiments, the macroporous compound component has a macroporous compound content of at least about 50 wt. %, at least about 60 wt. %, at least about 70 wt. %, at least about 80 wt. %, at least about 90 wt. %, or at least about 95 wt. %. For example, the macroporous compound component can have macroporous compound content from about 50 wt. % to about 99 wt. %, from about 60 wt. % to about 90 wt. %, or from about 80 wt. % to about 90 wt. %.

The extraction mixture also comprises a base. In some embodiments, the base can comprise a strong base. For example, the base can comprise sodium hydroxide and/or potassium hydroxide. The concentration of the base in the extraction mixture can be from about 1 mM to about 500 mM, from about 50 mM to about 250 mM, or from about 75 mM to about 150 mM. In various embodiments, the pH of the extraction mixture is no greater than about 13, no greater than about 12.5, or no greater than about 12. In some embodiments, the extraction mixture can have a pH from about 10 to about 13, from about 10 to about 12.5, from about 10 to about 12, from about 11 to about 13, from about 11 to about 12.5, or from about 11 to about 12.

The extraction mixture also comprises a solvent. For example, the solvent typically comprises water. In some embodiments, the solvent consists or consists essentially of water.

In some embodiments the extraction mixture further comprises an emulsifying agent. The emulsifying agent can comprise, for example, a nonionic surfactant or a cationic surfactant. In various embodiments, the emulsifying agent comprises a polysorbate, a quaternary ammonium surfactant or a cationic detergent. For example, the emulsifying agent can comprise TWEEN (e.g., TWEEN20) and/or CTAB.

In various embodiments, the extraction mixture further comprises lysis components that aid in the breakdown of these cellular or viral components and thus allow the release of polynucleotides into the extraction mixture. As used herein, lysis components can comprise detergents, emulsifying agents, surfactants, buffers, bases, chelators, or any combination thereof. In some embodiments, the basic mixture, as described herein, can comprise lysis components. For example, sample comprising particulates can be mixed with a basic mixture comprising a solvent, a base, and at least one lysis component that aids in the breakdown of cellular or viral components to release polynucleotides into the basic mixture.

The preparation of the extraction mixture can comprise a single step, or can occur in multiple steps. For instance, the sample comprising particulates can be mixed with the macroporous compound component, the base, and the solvent in a single step.

In some other embodiments, the macroporous compound component, sample, base and solvent can be premixed and combined in separate steps. For example, preparation of the extraction mixture can include mixing a first portion of the solvent with the sample and the base to form a basic mixture; obtaining a lysate from the basic mixture; and mixing the macroporous compound component with a portion of the lysate to form the extraction mixture. In some embodiments, the basic mixture comprises from about 1 mM to about 500 mM, from about 50 mM to about 250 mM, or from about 75 mM to about 150 mM of the base. In various embodiments, the pH of the basic mixture is no greater than about 13, no greater than about 12.5, or no greater than about 12. In some embodiments, the extraction mixture can have a pH from about 10 to about 13, from about 10 to about 12.5, from about 10 to about 12, from about 11 to about 13, from about 11 to about 12.5, or from about 11 to about 12.

Also, in various embodiments, the concentration of particulates in the basic mixture can be from about 20 wt. % to about 50 wt. %, from about 25 wt. % to about 45 wt. %, or from about 30 wt. % to about 35 wt. %. The amount of particulates present can be expressed in terms of a ratio of volume (in cm³) to volume (in mL) of soil to solvent. In these embodiments, the basic mixture can have a volumetric ratio of soil to solvent of from about 1:1 to about 1:4, or from about 1:2 to about 1:4 (e.g., about 1:3). In some embodiments, the ratio of the volume of the sample comprising particulates (cm³) to the volume (mL) of the basic mixture is from about 1:1 to about 1:4, from about 1:2 to about 1:4, or about 1:3.

In some embodiments, the extraction mixture can be prepared by mixing a macroporous compound component having a macroporous compound content from about 80 wt. % to about 90 wt. % in solvent with a portion of a lysate obtained from a basic mixture comprising about 75 mM to about 150 mM of a base and 25 wt. % to about 45 wt. % of particulates such that the volumetric ratio of the macroporous compound component to the lysate is about 1:1 to about 2:1, about 1.2:1 to about 1.8:1, or about 1.4:1 to about 1.6:1.

These multi-step methods can further comprise separating the basic mixture into a solid portion and a supernatant and obtaining the lysate from the supernatant. In general, the methods for separation comprise methods that can attract DNA or pull DNA out of solution. For example, in some embodiments, the separation can comprise filtration, membrane separation techniques, centrifugation, sedimentation, chelation (e.g., using magnetic beads), electromagnetic attraction (e.g., using a heated or electrically charged surface such as a wire) and combinations thereof.

In general, the methods described herein further comprise separating the extraction mixture into an extract comprising at least a portion of the polynucleotides and a first fraction comprising at least a portion of the macroporous compound component and the soil particles. The separation of the extraction mixture into the extract comprising polynucleotides and the first fraction can comprise, for example, filtration, a membrane separation technique, centrifugation, sedimentation, chelation, electromagnetic attraction or any combination thereof. In some embodiments, the methods do not include a filtration step. In certain embodiments, separation of the extract comprising polynucleotides from the extraction mixture is conducted via sedimentation alone or in combination with one or more other techniques.

In some embodiments, the first fraction comprising at least a portion of the macroporous compound component and the particulates comprises an inhibitor as described herein. In various embodiments, the concentration of the inhibitor in the extract is less than the concentration of the inhibitor in the first fraction comprising the particulates and the macroporous compound component. In some embodiments, the concentration of the inhibitor in the extract comprising polynucleotides is less than the concentration of the inhibitor in the extraction mixture.

The methods of the present invention advantageously provide for extracts that suitable for analysis. In various embodiments, the extract has enhanced optical properties, such as increased clarity or reduced turbidity, as compared to the sample, a dilution of the sample, and/or lysate as described herein. For example, in various embodiments, the extract can have an absorbance that is less than about 1, less than about 0.5, or less than about 0.2 when measured between about 400 and about 500 nm at 25° C. In some embodiments, the extract can have an absorbance that is less than about 1, less than about 0.5, or less than about 0.2 when measured at 492 nm at 25° C. Absorbance can be measured using a spectrophotometer.

### Methods for Analyzing Polynucleotides

The present invention also includes various methods for analyzing a polynucleotide. In general, the methods comprise preparing an extract comprising polynucleotides according to any of the methods described herein and analyzing, manipulating or using the polynucleotides in any application known to one skilled in the art to require polynucleotides.

For example, the methods can comprise preparing an extract comprising polynucleotides according to any of the methods described herein and detecting or identifying the polynucleotide in the extract.

Various methods of identifying or detecting a polynucleotide sequence in an extract are known in the art.

Exemplary methods for detecting or identifying the polynucleotide in the extract comprise amplifying the polynucleotide. Accordingly, various methods comprise preparing an extract comprising polynucleotides according to any of the methods described herein; and amplifying a polynucleotide in the extract.

The polynucleotide can be amplified using standard techniques available in the art. For example, the amplification procedure can comprise the polymerase chain reaction (PCR), multiplex PCR, a reverse transcriptase reaction (RT), loop mediated isothermal amplification (LAMP), nucleic acid sequence based amplification (NASBA), self-sustain sequence replication (3SR), strand displacement amplification, helicase-dependent amplification, nicking enzyme amplification reaction, multiple displacement amplification, rolling circle amplification, ligase chain reaction, ramification amplification method, or combinations thereof.

Advantageously, the amplification procedure can comprise an isothermal reaction. For example, the amplification procedure can comprise loop mediated isothermal amplification (LAMP), nucleic acid sequence based amplification (NASBA), self-sustain sequence replication (3SR), strand displacement amplification, or combinations thereof. Isothermal amplification methods, and their use in microfluidic devices, are described in the following, incorporated herein by reference: Zanoli, L. M., & Spoto, G. (2013). “Isothermal Amplification Methods for the Detection of Nucleic Acids in Microfluidic Devices.” Biosensors, 3(1), 18-43.

The amplification procedure can comprise PCR or variant thereof. For example, the amplification procedure can comprise allele-specific PCR, assembly PCR, asymmetric PCR, convective PCR, dial-out PCR, digital PCR, hot start PCR, intersequence-specific PCR, inverse PCR, ligation mediated PCR, methylation-specific PCR, miniprimer PCR, multiplex ligation-dependent probe amplification, multiplex PCR, nanoparticle assisted PCR, nested PCR, overlap-extension PCR, PAN-AC, RNase H-dependent PCR, single specific primer-PCR, solid phase PCR, thermal asymmetric interlaced PCR, touchdown PCR, universal fast walking or any combination thereof. Some amplification procedures (i.e., many of the standard PCR based reactions described herein) require a thermo-cycling step. Heating the reaction mixture denatures double stranded nucleotides and allows a polymerase to access the exposed nucleotide strand. In contrast, other “isothermal” techniques use other molecular approaches to denature double stranded polynucleotides. Various thermo-cycling dependent techniques (e.g., standard PCR assays) are described and compared to isothermal methods in the literature, see for example: Goda, Tatsuro, Miyuki Tabata, and Yuji Miyahara. “Electrical and Electrochemical Monitoring of Nucleic Acid Amplification.” Frontiers in Bioengineering and Biotechnology 3 (2015): 29.

In some embodiments, the methods of amplifying the polynucleotide can comprise a polymerase chain reaction and/or loop mediated isothermal amplification (LAMP).

The method of amplifying the polynucleotide can further comprise detecting the amplicon using any standard method in the art. For example, the amplified polynucleotide can be run on an agarose gel in the presence of an electric field. Standard nucleic acid staining agents can be used, as is understood in the art, to visualize the amplified polynucleotide in the gel.

In some embodiments, the method of amplifying a polynucleotide further comprises monitoring the amplification of the polynucleotide. The monitoring can use any standard method in the art. For example, the monitoring can comprise simple end-point PCR with gel visualization. In this embodiment, the polynucleotide is amplified and the amplicon (i.e the amplified polynucleotides) are run in the presence of an electric field on a gel substrate (e.g., agarose gel). This separates the amplicons by size and allows for an easy visual assessment of the presence of the target polynucleotide in the original sample. Other techniques are available in the art where the progress of an amplification reaction is tracked or monitored in real time (e.g., as the reaction is occurring). These real-time monitoring methods can comprise quantitative real time (qRT) PCR, quantitative LAMP, quantitative nucleic acid sequence-based amplification (QT-NASBA), digital PCR, or combinations thereof. Various detection/monitoring methods are further described in: Goda, Tatsuro, Miyuki Tabata, and Yuji Miyahara. “Electrical and Electrochemical Monitoring of Nucleic Acid Amplification.” Frontiers in Bioengineering and Biotechnology 3 (2015): 29. PMC. Web. 10 May 2018.

The monitoring of the amplification of the polynucleotide can comprise any technique known in the art that correlates the amount of polynucleotides with a fluorescent, luminescent or other optical signal. For example, the monitoring can use a small polynucleotide-interacting molecule such as a fluorescent probe or nucleic acid marker. As another example, the monitoring can use a nucleic acid binding dye (e.g., ethidium bromide) to detect the presence or absence of an amplified polynucleotide in a gel substrate (e.g., end-point PCR with gel).

In some embodiments, the fluorescent probe is a molecule comprising a fluorophore and a quencher that binds to a certain polynucleotides. As the polynucleotides are amplified the probe is cleaved, releasing the fluorophore from its quencher and allowing the emittance of a fluorescent signal. A suitable fluorescent probe that can be used in the methods herein includes the TAQMAN probe. In some embodiments, an amplification procedure comprising the polymerase chain reaction (PCR) can be monitored using a TAQMAN probe.

In some other embodiments, the amplification of the polynucleotide can comprise using a nucleic acid marker. Nucleic acid markers are small molecules that bind polynucleotides and the resulting marker-nucleotide complex emits a luminescent signal which is proportional to the amount of polynucleotides in a solution. A suitable nucleic acid marker that can be used in the methods herein includes SYBR-green. In some embodiments, an amplification procedure comprising the loop mediated isothermal amplification (LAMP) can be monitored using SYBR-green.

In some embodiments, the polynucleotide comprises RNA and the methods further comprise reverse transcribing the RNA into a cDNA transcript, using standard methods in the art. In some embodiments, the cDNA transcript can be amplified. In some embodiments, the amplification of the cDNA transcript can be monitored using methods known in the art.

In some embodiments, the methods of analyzing the polynucleotide can comprise sequencing the polynucleotide using any standard method in the art. Traditional and next generation sequencing methods are described in the following, non-limiting references, each incorporated herein by reference: Mardis ER “Next-generation DNA sequencing methods”Annu Rev Genomics Hum Genet (2008) 9:387-402; El-Metwally S., Ouda O. M., Helmy M. (2014) New Horizons in Next-Generation Sequencing. In: Next Generation Sequencing Technologies and Challenges in Sequence Assembly. SpringerBriefs in Systems Biology, vol 7. Springer, New York, N.Y.; Pareek, C. S., Smoczynski, R., & Tretyn, A. (2011). Sequencing technologies and genome sequencing. Journal of Applied Genetics, 52(4), 413-435; Di Bella J M, Bao Y, Gloor B. B., Burton J. P, & Reid G. “High throughput sequencing methods and analysis for microbiome research” J. Microbiological Methods (2013) 95(3):401-414; Wang, Y., Yang, Q., & Wang, Z. (2014). The evolution of nanopore sequencing. Frontiers in Genetics, 5, 449.

For example, sequencing the polynucleotide can comprise traditional sequencing methods like chain termination (eg., Sanger sequencing or Maxam-Gilbert sequencing) or can comprise next generation sequencing (NGS) techniques (e.g., single molecule real-time sequencing, ion semiconductor sequencing, pyrosequencing (ROCHE 454 FLX), sequence by synthesis (ILLUMINA), emulsion PCR (SOLiD platform, Applied Biosystems), nanopore sequencing (MinION, Oxford Nanopore Technologies), RNA-sequencing (RNA-Seq). In some embodiments the sequencing the polynucleotide comprises Sanger sequencing, pyrosequencing, sequence by synthesis, emulsion PCR, or nanopore sequencing. As understood in the art, sequencing the polynucleotide may or may not comprise amplifying the polynucleotide. For example, traditional methods like chain termination do require polynucleotide amplification, but next generation methods (e.g., nanopore sequencing) do not. The polynucleotide in the extract prepared using the methods herein can be ideally sequenced using any methods known in the art, with or without amplification.

It should also be noted that the methods of analyzing, manipulating, or otherwise using the polynucleotide extract prepared herein should not be limited to the methods explicitly described herein. To this end, any technique available to one skilled in the art when practicing the invention is envisioned for the present invention. These techniques can include but are not limited to: genome editing, transformation, gene therapy, cellular delivery of nucleic acids, or any other techniques that require a polynucleotide.

### Methods for Detecting an Organism or Agent Comprising a Polynucleotide in a Sample

The present invention further includes various methods for detecting an organism or agent that comprises a polynucleotide in a sample. The methods comprise: preparing an extract comprising polynucleotides from the sample according to any of the methods described herein, analyzing a polynucleotide obtained from the extract, and detecting the organism or agent in the sample based on the analysis of the polynucleotide.

The polynucleotide can be analyzed using any method described herein or known in the art. For example, the polynucleotide can be identified by amplifying and/or sequencing the polynucleotide. In some embodiments, the polynucleotide can be detected by amplification using a polymerase chain reaction (PCR) or loop mediated isothermal amplification (LAMP).

Once analyzed, the polynucleotide can then identify an organism or agent that comprises a polynucleotide in the sample using standard techniques known in the art. An agent that comprises a polynucleotide generally refers to an inanimate entity that comprises or is complexed with genetic material (e.g., a polynucleotide). For example, an agent that comprises a polynucleotide can comprise a virus, a viroid, a virion, or any combination thereof. An organism that comprises a polynucleotide generally refers to an animated entity that comprises genetic material. For example, the organism can comprise a microbe, bacteria, fungus, oomycetes, protozoa, phytoplasma, and/or a plant. The organism can be a microbe defined herein to be a single celled or multicellular microscopic living organism (i.e., a microorganism). Microorganisms are diverse and include all the bacteria, archaea, protozoa, fungi and algae, especially cells of plant pathogens. Certain animals are also considered microbes (e.g., rotifers).

In various embodiments, a microbe can be any of several different microscopic stages of a plant or animals. Microbes can also include viruses, viroids, and prions, especially those which are pathogens or symbiots to crop plants. In other embodiments, the organism can be macroscopic. For example, the organism can comprise a fungus, a nematode, an insect, a plant or plant part (e.g., a seed), or any other plant or animal that can be identified in a growing area.

The organism or agent detected by the methods described herein can be any organism or agent that affects plant health (e.g., any organism whose presence near, on, or in a plant is associated with changes in the growth, development, or performance of the plant). In some embodiments, the association of a plant with the detected organism or agent is beneficial for the plant. For example, the organism or agent can result in an increase or improvement of the growth survival, reproduction, or productivity of the at least one plant.

In other embodiments, the organism or agent can be harmful to the at least one plant (e.g., can comprise a pathogen). The pathogen can be, for example, selected from the group consisting of microorganisms, viruses, nematodes, fungi, bacteria, oomycetes, protozoa, phytoplasma, parasitic plants, insects, mites, gastropods, arthropods, moths, thrips, locusts, crickets, beetles, worms and combinations thereof. Other pathogens include nematodes, insects, or viruses. In certain embodiments, the pathogen hinders the growth survival, reproduction, or productivity of at least one plant.

As used herein, the fungus or fungi can comprise a whole fungus, any part thereof, or a cell or tissue culture derived from a fungus, comprising any of: whole fungus, fungus components or organs, fungal tissues, spores, fungal cells, including cells of hyphae and/or cells of mycelium, and/or progeny of the same. A fungus cell is a biological cell of a fungus, taken from a fungus or derived through culture from a cell taken from a fungus.

In still other embodiments, the organism or agent detected can be neutral (i.e., not noticeably affect the growth, survival, reproduction, or productivity of the plant).

### Methods for Identifying and/or Quantifying Soil Organisms that Affect Plant Health

The method of detecting an organism or agent comprising a polynucleotide can be directed towards identifying soil organisms in a sample. In this embodiment, the soil organism is any organism whose presence near, on or in a plant is associated with changes in the growth, development or performance of the plant. In some embodiments, the method of detecting or identifying a microbial soil organism can comprise detecting a plurality of soil organisms. For example, a plurality of samples can be taken from a growing area (e.g., a field or growing chamber), and each used to provide a polynucleotide extract which can be, in turn, used to identify populations of microbial soil organisms in the growing area. In some embodiments, a map of a plurality of soil organisms, their locations, and quantity, can be generated for a growing area (e.g., a field). This map can be monitored with different treatment programs (E.g., pesticide application) to determine the effectiveness of a given treatment on the microbial soil ecosystem in the growing area.

In some embodiments, the method of detecting an organism or agent further comprises using an Infection Index Method to quantify the number of target organisms in a sample of matter. The Infection Index Method comprises comparing the amount of nucleic acids detected with a sequence specific to a target organism to the total amount of nucleic acids detected in the sample of matter. The Infection Index Method is described in U.S. Pat. No. 8,686,219, which is hereby incorporated by reference in its entirety.

### Methods for Detecting the Presence of Plant DNA Sequences in a Population of Plants, Growing Area, or Soil Sample

The methods of the present invention can also be directed towards detecting the presence of a target plant nucleic acid in a population of plants, growing area or soil sample. In certain embodiments, the methods comprise detecting transgenes (e.g., nucleic acid sequences not native to a plant) in the growing plant, soil, or residual (e.g., post-harvest) plant tissues in a growing area.

The plant or population of plants can comprise a crop plant. As used herein a crop plant may be any plant grown or cultivated for human and/or animal use. For example, a crop plant can comprise a plant that produces edible tissues and/or other plant products like grains (e.g., wheat, corn), legumes (e.g., soybeans, dry beans, snap beans), tuberous plants (e.g., potatoes), vegetables (e.g., squash, stalk plants), oilseeds (e.g. canola, soybeans), and/or fruits (e.g., citrus crops) or it can comprise any industrial crop grown to produce non-edible goods for manufacturing (e.g. cotton and/or hemp fiber, wheat biopolymers, etc.), pharmaceuticals (e.g. Artemisia artemisinin, etc.), etc. For example, in various embodiments, the crop plant comprises corn, soybean, cotton, peanuts, potatoes, canola, sugarbeets, grain sorghum (milo), field beans, or a combination thereof. In some embodiments, the crop plant is selected from Amaranthaceae (e.g., chard, spinach, sugar beet, and quinoa); Amaryllidaceae (e.g., chives, bulb onion, garlic, green onion, leeks, and shallot); Apiaceae (e.g., anise, caraway, carrot, celery, chervil, coriander, cumin, dill, fennel, parsley, and parsnip); Asparagaceae (e.g., agave and asparagus); Asteraceae (e.g., artichoke, asters, chamomile, chicory, chrysanthemums, dahlias, daisies, echinacea, goldenrod, guayule, lettuce, marigolds, safflower, sunflowers, and zinnias); Brassicaceae (e.g., arugula, broccoli, bok choy, Brussels sprouts, cabbage, cauliflower, canola, collard greens, daikon, garden cress, horseradish, kale, mustard, radish, rapeseed, rutabaga, turnip, wasabi, watercress, and Arabidopsis thaliana); Bromeliaceae (e.g., pineapple), Convolvulaceae (e.g., morning glory and sweet potato); Cucurbitaceae (e.g., cantaloupe, cucumber, honeydew, melon, pumpkin, watermelon, zucchini, and squash such as acorn squash, butternut squash, and summer squash); Fabaceae (e.g., alfalfa, beans, carob, clover, guar, lentils, mesquite, peas, peanuts, soybeans, tamarind, tragacanth, and vetch); Malvaceae (e.g., cacao, cotton, durian, hibiscus, kenaf, kola, and okra); Musaceae (e.g., banana and plantain); Poaceae (e.g., bamboo, barley, corn, fonio, millet, oats, ornamental grasses, rice, rye, sorghum, sugar cane, triticale, wheat, and lawn grasses such as Bahia grass, Bermudagrass, bluegrass, Buffalograss, Centipede grass, Fescue, or Zoysia); Polygonaceae (e.g., buckwheat); Rosaceae (e.g., almonds, apples, apricots, blackberry, blueberry, cherries, peaches, plums, quinces, raspberries, roses, and strawberries); Rubiaceae (e.g., coffee); Solanaceae (e.g., bell peppers, chili peppers, eggplant, petunia, potato, tobacco, and tomato); Vitaceae (e.g., grape), and combinations thereof.

As used herein the term plant product will be understood. to mean the product derived from or produced by a plant. For example, plant product can comprise the tissues or structures of the plant such as the flower, fruit, seed, grain, leaves, stems etc., produced by the plant. For example, seed cotton (or cotton bolls) from cotton plants, corn from corn plants, soy beans from soy plants, canola seeds from canola plants, wheat grain from wheat plants, or the leaves, stems, vegetables, seeds, grains, etc., from any other plant can all be considered to be a “plant product”.

Other embodiments of the invention comprise evaluating a population of plants for certain genetic traits, alleles, or sequences (e.g., in a breeding program). As used herein the phrase population of plants or plant population means a set comprising any number, including one, of individuals, objects, or data from which samples are taken for evaluation, e.g. estimating quantitative trait locus (QTL) effects and/or disease tolerance. Most commonly, the terms relate to a breeding population of plants from which members are selected and crossed to produce progeny in a breeding program. A population of plants can include the progeny of a single breeding cross or a plurality of breeding crosses, and can be either actual plants or plant derived material, or in silica representations of the plants. The population members need not be identical to the population members selected for use in subsequent cycles of analyses. The evaluation of the plant population can comprise preparing a polynucleotide extract or extracts from samples taken from the plant population and analyzing the polynucleotides therein for alleles, or genetic traits linked to crop or plant performance or tolerance to disease conditions.

As used herein, crop or plant performance is a metric of how well a crop plant grows under a set of environmental conditions and cultivation practices. Crop/plant performance can be measured by any metric a user associates with a crop's productivity (e.g., yield), appearance, and/or robustness (e.g., color, morphology, height, biomass, maturation rate), product quality (e.g., fiber lint percent, fiber quality, seed protein content, seed carbohydrate content, etc.), cost of goods sold (e.g., the cost of creating a seed, plant, or plant product in a commercial, research, or industrial setting) and/or a plant's tolerance to disease (e.g., a response associated with deliberate or spontaneous infection by a pathogen) and/or environmental stress (e.g., drought, flooding, low nitrogen or other soil nutrients, wind, hail, temperature, day length, etc.). Crop/plant performance can also be measured by determining a crop's commercial value and/or by determining the likelihood that a particular inbred, hybrid, or variety will become a commercial product and/or by determining the likelihood that the offspring of an inbred, hybrid, or variety will become a commercial product. Crop/plant performance can be a quantity (e.g., the volume or weight of seen or other plant product measured in liters or grams) or some other metric assigned to some aspect of a plant that can be represented on a scale (e.g., assigning a 1-10 value to a plan based on its disease tolerance).

Therefore, in one embodiment, a plant or plant population may be exposed to a disease condition, and the tolerance or resistance of the plant or plant population determined. Thus, in some embodiments, the method of detecting the organism or agent further comprises phenotyping a plant for tolerance to a pathogen.

As used herein, the term tolerance or improved tolerance in a plant to disease conditions will be understood to mean an indication that the plant is less affected by disease conditions with respect to yield, survivability, and/or other relevant agronomic measures, compared to a less tolerant, more “susceptible” plant. Tolerance is a relative term, indicating that a “tolerant” plant survives and/or produces better yields in disease conditions compared to a different (less tolerant) plant (e.g., a different corn line strain) grown in similar disease conditions. As used in the art, disease “tolerance” is sometimes used interchangeably with disease “resistance”. One of skill in the art will appreciate that plant tolerance to disease conditions varies widely and can represent a spectrum of more-tolerant or less-tolerant phenotypes. However, by simple observation, one of skill in the art can generally determine the relative tolerance or susceptibility of different plants, plant lines, or plant families under disease conditions, and furthermore, will also recognize the phenotypic gradations of “tolerant”.

In various embodiments, the methods of detecting an organism or agent can further comprise identifying an allele or quantitative trait locus (QTL) of a plant that is associated with disease tolerance. In some embodiments, an extract comprising polynucleotides can be prepared from the plant or plant populations exposed to a disease condition, using any method described herein, and analyzed to identify an allele or quantitative trait locus (QTL) that may be linked to the given tolerance or resistance (or lack thereof) in the plant or plant population to the disease condition. In still further embodiments, the plant population can be used in a breeding program to select for or enhance favorable traits that confer increased tolerance or resistance to the disease condition. Therefore, in some embodiments the methods of detecting the organism or agent can comprise determining whether a plant should be chosen as a parent in a breeding program, based on plant performance or tolerance to a disease condition.

In further embodiments, methods are provided for selecting a plant for advancement in a breeding program. The methods comprise preparing an extract comprising polynucleotides using any method described herein, analyzing a polynucleotide obtained from the extract to assign a genotype to a plant, and selecting for advancement in a breeding pipeline a plant based on the analysis of the polynucleotide. In some embodiments, the analysis comprises any method of analysis described herein. The genotype identified or assigned to the plant can be a genotype linked in any way to the plant's performance. The plant's performance is as described herein and can include, but is not limited to, measurements of the plant's growth, reproducibility, yield, and/or pest/agrochemical/disease tolerance.

Likewise, in further embodiments, the soil around a plant, including the rhizosphere, can be sampled and a polynucleotide extract prepared therefrom. The microbial soil organisms present in the soil sample can be identified and/or quantified as described herein, e.g., by use of the Infection Index Method. The level of microbial soil organisms in the sample can then be used to evaluate plant tolerance or otherwise determine crop or plant performance of the plant.

### Methods for Treating a Plant or Seed

The present invention further includes methods for treating a plant or plant part (e.g., a seed). Various methods comprise: preparing an extract comprising polynucleotides using any method described herein, analyzing a polynucleotide obtained from the extract and applying to the plant or plant part, a treatment based on the analysis of the polynucleotide that improves plant performance.

In various embodiments the plant or plant part that is treated is or is derived from a crop plant, as defined herein.

In certain embodiments, the extract comprising polynucleotides is prepared from a soil sample taken from the growing area of the plant or plant part receiving the treatment. In other embodiments, the extract is prepared from a soil sample not taken from the growing area of the plant or plant part receiving the treatment.

The polynucleotide can be analyzed using methods known in the art. In some embodiments, the polynucleotide can be analyzed using various methods described herein. For example, the polynucleotide can be amplified using the polymerase chain reaction (PCR) or loop mediated isothermal reaction (LAMP). Alternatively, or in addition, the polynucleotide can be sequenced using, for example, Sanger sequencing or nanopore sequencing.

In some embodiments, the methods comprise treating the plant against a pathogen. In some embodiments, the pathogen is identified and/or detected in the sample by the analysis of the polynucleotide. In further embodiments, the pathogen is quantified based on the quantity of the polynucleotide in the sample.

In other embodiments, the methods comprise treating the plant with a beneficial organism (e.g., bacteria). The beneficial organism may be identified based on the analysis of the polynucleotide. Further, the beneficial organism can be quantified in the sample based on the quantity of the polynucleotide in the sample. In some embodiments, the plant or plant part receiving the treatment may be located in the same growing area that sourced the sample. In other embodiments, the plant receiving the treatment may be located in a different growing area that sourced the sample.

In various embodiments, the treatment can be chosen based on the analysis of the polynucleotide. Preferably, the treatment is chosen based on the identity, detection, and quantity of the beneficial organism or pathogen based on the analysis of the polynucleotide. For example, if a pest (e.g., a nematode) is detected, the treatment can comprise a pesticide (nematicide). As another example, if a beneficial organism is detected, it can be applied directly on another population of plants to improve their performance.

In some embodiments, the treatment can comprises an agrochemical, an organism (e.g., a beneficial organism as described herein), a viral vector or a transection/transformation agent. Various agrochemicals comprise various pesticides such as nematicides, herbicides, fungicides, insecticides, antibiotics, antimicrobials, as well as other soil amendments such as fertilizers and any combination thereof. For example, the agrochemical can comprise a nematicide and/or a fungicide.

### High Throughput Methods

Methods disclosed herein can be used in conjunction with a wide range of soil sampling methods that allow users to obtain a plurality of soil samples from a growing area. In addition, automated methods can be used to extract and, optionally, analyze polynucleotides from the samples in a high-throughput manner.

Automated soil sampling methods include mobile devices capable of traversing a growing area and that extract a plug or core of soil at some desired frequency. For example, such systems can comprise a hollow core or plug sampling tube that extends from an outer edge of a rotating wheel or track and that is driven into the soil each time the rotation bring the tube into contact with the soil. The tube is removed from the soil as the system traverses the field and the plug or core of soil within the tube is removed and placed into a container. In some embodiments, the extraction methods described herein could be applied to a sample of soil collected this way to analyze (e.g., by amplifying) polynucleotides within the soil. Automated methods of adding the extraction reagents described herein and subjecting the combination to rapid nucleic acid amplification to quantify the presence of certain organisms in the soil are further envisioned, e.g. to rapidly quantify the amount of pests in a growing area.

Any automated soil collecting device may be used in the present invention. Automated soil collectors can include mobile plug/core samplers or can include “rotating” soil sampling systems. Examples of mobile plug/core samplers can include the Big John Speedy soil sampler, various models from Amity Technology, various models from Wintex Agro, and the GVM Agriprobe. Examples of “rotating” soil sampling systems include the Falcon 5000 from Falcon Soil Technologies and the AutoProbe from AgRobotics.

Methods described herein are not limited to use with soil samplers that remove a plug or core of soil. For example, a blade connected to a mobile platform could be inserted to a desired depth into the surface of a field such that when the mobile platform traverses the field, the blade is drug through the soil, exposing soil that was previously below the surface of the field. In certain embodiments, this exposed soil could be sampled with a simple scoop that diverts a portion of the soil into a container where the nucleic acids could be extracted and amplified using the methods described herein.

In certain embodiments, the methods described herein comprise obtaining the sample from soil that is exposed in the furrow during planting. An auger or diverter placed near the opening/closing disks of a planter head, for example, can be used to direct a portion of the soil exposed during planting into a container where the nucleic acids could be extracted and amplified using the methods describe herein. U.S. Pat. No. 7,216,555, which is incorporated by reference herein, describes a variation of a soil collection method wherein a “shoe” cuts a horizontal slab of soil and then replaces it. Various embodiments of the method described herein can comprise collecting soil samples from this horizontal slab of soil.

In various embodiments, the soil sample collection device further comprises one or more arms connected to the mobile platform, each arm can be extendable and/or retractable with respect to the mobile platform to collect the soil sample. In some embodiments, the mobile arms can allow for efficient soil collection in an interior location (e.g., a greenhouse). In some embodiments, the mobile platform is positioned above the soil samples (e.g., on a ceiling in a greenhouse) and the mobile arms directed to obtain the plurality of soil samples for analysis. In these embodiments, any of the soil collecting devices described herein (i.e., an auger, digger, diverter, etc.) may be attached to the mobile arms to facilitate the soil collection from this type of platform.

In various embodiments, soil samples collected using these high throughput/automated methods can be diverted to a container where polynucleotides can be extracted and amplified using the methods described herein. Preferably, the container and reagents for extraction and amplification can be configured on to the collecting device to allow for on-site, high throughput, data analysis of soil samples on the field. For example, U.S. Patent Application Publication 2017/00223947, which is incorporated by reference herein, describes a functional device capable of obtaining soil samples and performing analysis. The polynucleotide extraction and amplification methods described herein are optimal for use in such a device.

In various embodiments, once the extract comprising polynucleotides is prepared, the method can further comprise diverting it into such a portable device to allow for on-site analysis. Suitable devices can include a microfluidic device configured for nucleic acid amplification, particularly using isothermal methods, such as described in Zanoli, L. M., & Spoto, G. (Isothermal Amplification Methods for the Detection of Nucleic Acids in Microfluidic Devices. (2013) Biosensors, 3(1), 18-43), which is incorporated by reference herein. Other devices, particularly suited for on-site sequencing or amplification of nucleic acids can include the MinION device (Oxford Nanopore Technologies), FREEDOM4 (Otago Innovation), or the TWO3 Real-Time PCR Thermocycler (Biomeme Inc).

The high throughput methods described herein can be performed using a mobile soil analysis system or mobile soil treatment system. Suitable systems are described in U.S. Patent Application Publication 2017/00223947

### Mobile Soil Analysis System

The present invention also includes various apparatus for performing the methods described herein. For example, various apparatus include a mobile soil analysis system. In some embodiments, the mobile soil analysis system comprises: a soil sample collection device; at least one vessel sized and shaped to receive the soil sample and one or more analysis reagents; a polynucleotide detector configured to receive at least a portion of the soil sample and one or more analysis reagents from the at least one vessel and identify and/or quantify polynucleotides in the soil sample, wherein the polynucleotide detector is configured to generate a polynucleotide signal; a soil sample processor in communication with the polynucleotide detector and configured to analyze the soil sample at least in part based on the polynucleotide signal; a tangible storage medium storing soil sample analysis instructions executable by the soil sample processor, wherein when the soil sample analysis instructions are executed by the soil sample processor, the polynucleotide signal is processed and the analytic data associated with the soil sample is stored on the tangible storage medium; and a mobile platform supporting the at least one vessel and the polynucleotide detector.

In some embodiments, the soil sample collection device can comprise an auger, diverter, bore, plug or any combination thereof

The polynucleotide detector can comprise a polynucleotide sequencer and/or amplifier. In some embodiments, the polynucleotide detector comprises a portable polynucleotide sequencer and/or amplifier. In some cases, the amplifier can comprise a microfluidic device configured for nucleic acid amplification, particularly using isothermal methods, such as described in Zanoli, L. M., & Spoto, G. (Isothermal Amplification Methods for the Detection of Nucleic Acids in Microfluidic Devices. (2013) Biosensors, 3(1), 18-43) incorporated herein by reference. Alternatively, or in addition, the polynucleotide detector can comprise a device suited for on-site sequencing or amplification of nucleic acids such as the MinION device (Oxford Nanopore Technologies), FREEDOM4 (Otago Innovation), or the TWO3 Real-Time PCR Thermocycler (Biomeme Inc).

The polynucleotide detector can also comprise a thermocycler as may be required for temperature dependent amplification methods (e.g., PCR). The polynucleotide signal can be an optical, luminescent and/or fluorescent signal. Therefore, the polynucleotide detector can also comprise a spectrophotometer, fluorimeter, light meter, or other optical detection device configured to detect the polynucleotide signal.

In some embodiments, the mobile soil analysis system can comprise a plurality of vessels, each vessel sized and shaped to receive a respective soil sample and one or more analysis reagents. In some embodiments, the mobile soil analysis system further comprises one or more agitators in fluid communication with the one or more vessels configured to mix the soil sample and one or more analysis reagents. In some embodiments, the mobile soil analysis system further comprises one or more containers for receiving one or more analytical reagents, wherein the containers are in fluid communication with the one or more vessels.

In some embodiments, the mobile soil analysis system is a high throughput system.

In various embodiments, the soil analysis system is a mobile soil analysis system structured and operable to traverse over and through a growing area. In various embodiments, the system can aerially traverse the growing area (e.g., using a drone or by using robotic arms suspended above the growing area, such as in a greenhouse). In various embodiments, the system can traverse the surface of the growing area (e.g., using a truck or other vehicle that drives on the surface of a field). Suitable systems configured to traverse a growing area and that can be modified according to the methods described herein are described in U.S. Patent Application 2017/0223947, U.S. Pat. No. 9,495,597, and U.S. Pat. No. 10,303,944, each of which is incorporated herein by reference in their entirety.

In some embodiments, the mobile soil analysis system further comprises an extraction conduit for removing at least a portion of the soil sample and one or more analysis reagents from the at least one vessel and wherein the extraction conduit is in fluid communication with the polynucleotide detector.

In further embodiments, the mobile soil analysis system further comprises one or more arms connected to the mobile platform. In various embodiments, each arm is extendable and/or retractable with respect to the mobile platform to collect the soil sample.

### Mobile Soil Treatment System

The present invention also includes various soil treatment systems. In various embodiments, soil treatment system is a mobile soil treatment system comprising: the mobile analysis system described herein, a container for receiving an agrochemical formulation; a dispenser for administering the agrochemical formulation to a soil collection, a growing area, a plant, a plant part, and/or locus thereof, wherein the dispenser is in fluid communication with the container.

In some embodiments, the dispenser is in communication with the soil sample processor and configured to dispense the agrochemical formulation based on the analytic data stored on the tangible storage medium. The dispenser can include an agrochemical formulation applicator and an applicator support constructed to support the agrochemical formulation applicator, the support supported by the mobile platform and movable with respect to the mobile platform to position the applicator for administering the agrochemical formulation.

The applicator support can comprise an arm connected to the mobile platform, the arm being extendable and/or retractable with respect to the mobile platform to position the applicator for administering the agrochemical formulation.

Having described the invention in detail, it will be apparent that modifications and variations are possible without departing from the scope of the invention defined in the appended claims.

### EXAMPLES

The following non-limiting examples are provided to further illustrate the embodiments(s) thereof, the articles “a”, “an”, “the” and “said” are intended to mean that there are one or more of the elements. The terms “comprising”, “including” and “having” are intended to be inclusive and mean that there may be additional elements other than the listed elements

### Example 1

### A Laboratory-Based Method of Extracting DNA from Soil Samples

This example describes a method suitable for use in a laboratory wherein a macroporous compound (e.g., macroporous resin) is used to purify soil samples to produce DNA that is selectively amplifiable.

### Soil Preparation

Several cups of soybean cyst (SCN) negative soil were removed from a field and placed into a bag for transport to a laboratory. A series of 100 cm³ (cc) samples from this soil were removed from the bag and placed into 250 mL bottles. Each soil sample (100 cc) was then inoculated with 0 to 40,000 SCN eggs, as indicated on Table 1, and then placed, without lid, into a −50° C. freezer for approximately 20 minutes. The bottles containing the inoculated 100 cc soil samples were removed from −50° C. and placed (without lid) in a dryer box until the soil appeared dry (usually at least two days, depending on the moisture level in the soil sample—sample needed to be dry enough that it will break up during the subsequent shaking/pulverizing steps). Once dry, two 0.75 inch (1.9 cm) steel BBs were added to each bottle and the bottle covered with a lid. The bottle was then placed into a modified paint can shaker that is standard in the art for breaking up soil samples. The bottle was then shaken to pulverize the sample for approximately 15 minutes and the BBs removed.

TABLE 1
SCN Eggs inoculated/100 cc of soil

| | |
|---|---|
| 0 | 5,000 |
| 50 | 7,500 |
| 100 | 10,000 |
| 250 | 12,500 |
| 500 | 15,000 |
| 750 | 17,500 |
| 1,000 | 20,000 |
| 1,250 | 22,250 |
| 1,500 | 25,000 |
| 1,750 | 27,500 |
| 2,000 | 30,000 |
| 3,000 | 35,000 |
| 4,000 | 40,000 |

Approximately 1 cc of crushed garnet mineral powder was placed into a 15 mL “lysis” vessel. A sufficient portion (about 3 ccs) of pulverized soil from each bottle was added to the 15 mL tube containing the garnet to bring the total volume of the vessel contents up to 4 mL. At this point, the lysis vessel contained approximately 3 cm³ (cc) of pulverized soil and 1 cm³ (cc) of garnet. Measurements from a few fields showed that the range of soil weight for this 3 cc volume of soil was around 2.7 g±0.38 g to 3.95±0.34 g (see Table 2, below). Two 0.25 inch (0.64 cm) BBs were then added to the lysis vessel, which was now ready for addition of the wet reagents of the lysis steps. This step was repeated for the series of soil samples inoculated with increasing concentrations of nematode eggs.

TABLE 2

| Field | Weight (g) | | |
|---|---|---|---|
| Identifier | 3 cc soil | Ave | STDEV |
| 501 | 2.78 | 2.74 | 0.38 |
| | 3.1 | | |
| | 2.34 | | |
| 441 | 2.97 | 3.10 | 0.11 |
| | 3.17 | | |
| | 3.15 | | |
| 511 | 3.67 | 3.95 | 0.34 |
| | 3.86 | | |
| | 4.33 | | |

### Cell Lysis

9 mL of lysis buffer comprising 100 mM NaOH and 2% TWEEN20 (polysorbate) was added to each lysis vessel (containing the soil, garnet, and BBs) and the lysis vessel placed into a paint can shaker for 12 minutes. After shaking/pulverizing, the lysis vessel was incubated at 95° C. for 30 minutes, and then returned to the shaker for 12 minutes. The lysis vessels were removed from the shaker and centrifuged at 2800 g for 20 minutes.

### DNA Extraction

A DAX-8 “extraction solution” was prepared comprising 5 g of DAX-8 suspended in 6 mL of water. Once the extraction solution was prepared, 0.5 mL of the extraction solution was placed into a series of extraction vessels (one for each lysis vessel, above). The lysis vessels were removed from the centrifuge and 0.3 mL of the lysate was removed from each lysis vessel and added to the corresponding extraction vessel containing the 0.5 mL DAX-8 extraction solution. The extraction vessel was then capped and inverted several times by hand to homogenize the solution, and then left undisturbed for at least 5 minutes. The extraction vessel was centrifuged at 2800g for 5 minutes. The lysate of this centrifugation step became the “1× lysate”. Therefore, after the soil lysis and extraction procedures a series of 1× lysates originating from samples spiked with 0 to 40,000 eggs/100 cc was generated. After centrifuging the extraction vessel, 50 μL of the 1× lysate was removed and diluted with 1×Tris-EDTA (TE) buffer to 0.01×. The diluted extracts were then subjected to PCR (TAQMAN) or LAMP amplification protocols using standard methods known in the art.

Amplification Set-Up: For the TAQMAN (PCR) protocol, 4 μL of the 0.01× DNA extract was transferred from a 96 well plate into a 384 well qPCR plate along with 6 μL of a SCN marker Master Mix comprising: 2× TAQMAN GTXPRESS MasterMix (Applied Biosystems), 1004 each forward and reverse SCN primers, and 1004 SCN probe (Table 3).

For LAMP, 5 μL of the 0.01× DNA extract was transferred along with 20 μl of a SCN marker Master Mix comprising: 1× Optigene Isothermal Master Mix (ISO-001) (Optigene LTD), 100 μM of each LAMP primer (SCN-FIP, SCN-BIP, SCN-F3, SCN-B3, SCN-LF, and SCN-LB), and water (Table 4). Each plate was transferred to a thermocycler programmed for PCR or LAMP amplification, as described in Table 5 below.

TABLE 3
TAQMAN Marker MasterMix

| | | Quantity | Quantity (μl) |
|---|---|---|---|
| | Concentration | (μL) per 300 | (pp |
| | (μM) | samples | per 1 sample |
| Lysate | 0.01× | — | 4 |
| TAQMAN | Manufacture | 1440 | 4.8 |
| GTXpress | (2×) | | |
| MasterMix | | | |
| SCN Forward | 10 | 160 | 0.53 |
| SCN Reverse | 10 | 160 | 0.53 |
| SCN Probe | 10 | 160 | 0.53 |

| | | |
|---|---|---|
| Total | 1920 | 10.4 |

TABLE 4
LAMP Marker Master Mix

| | Concentration | Quantity (μL) | Quantity (μl) |
|---|---|---|---|
| | (μM) | per 300 samples | per 1 sample |
| Lysate | 0.01× | — | 5 |
| Optigene Isothermal | Manufacture (1×) | 4500 | 15 |
| Master Mix ISO-001 | | | |
| Water | N/A | 1290 | 4.3 |
| SCN-FIP | 100 | 60 | 0.2 |
| SCN-BIP | 100 | 60 | 0.2 |
| SCN-F3 | 100 | 15 | 0.05 |
| SCN-B3 | 100 | 15 | 0.05 |
| SCN-LF | 100 | 30 | 0.1 |
| SCN-LB | 100 | 30 | 0.1 |

| | | |
|---|---|---|
| Total | 6000 | 25 |

TABLE 5

| | Temperature | Time | |
|---|---|---|---|
| | (° C.) | (sec) | Cycles |
| LAMP | 65 | 15 | 1 |
| | 65 | 15 | 240 |
| | 5 | 20 | 1 |
| TAQMAN | 95 | 1 | 45 |
| | 60 | 20 | |

### Data Analysis and Interpretation

Each amplification protocol was run with a series of standard controls containing known amounts of SCN transcript. Once the amplification cycles were complete, the Ct values for each standard were identified and used to generate a standard curve and a standard curve equation (y=1e−7x−8e−5). This standard curve equation was then used to calculate the quantity of SCN transcript in each soil lysate sample. Each egg count/100 cc generated 15 samples which were then used to determine the percentage of samples that returned usable data.

Table 6, below, depicts the % data return across the 15 samples/egg count that achieved usable data and the average DNA quantity detected in each LAMP experiments using Rapid Soil Pathogen Genotyping Version 2.0 (no DAX-8) and Rapid Soil Pathogen Genotyping Version 3.0 (with DAX-8). It is clear from the data depicted that amplification was observed reliably at around 750 eggs/100 cc for experiment run with DAX and that the average DNA quantity correlated with the number of eggs in the presence of DAX (but not in the lack of DAX).

TABLE 6

| | Rapid Soil Pathogen | | | Rapid Soil Pathogen | | |
|---|---|---|---|---|---|---|
| | Genotyping NO DAX-8 | | | Genotyping WITH DAX-8 | | |
| | | Average DNA | | | Average DNA | |
| Eggs/ | % Data | Quantity (pg) | | % Data | Quantity (pg) | |
| 100 cc | Return | Avg | Stdev | Return | Avg | Stdev |
| 0 | 13% | 9.97 | 14.10 | 8% | 0.00 | 0.00 |
| 100 | 0% | 0.00 | 0.00 | 80% | 0.00 | 0.00 |
| 250 | 0% | 0.00 | 0.00 | 87% | 0.04 | 0.12 |
| 500 | 0% | 39.90 | 0.00 | 87% | 0.02 | 0.02 |
| 750 | 7% | 3.41 | 0.00 | 100% | 0.02 | 0.02 |
| 1,000 | 40% | 41.50 | 80.60 | 100% | 0.05 | 0.04 |
| 2,500 | 53% | 0.54 | 1.32 | 93% | 0.18 | 0.17 |
| 5,000 | 60% | 4.00 | 4.86 | 93% | 0.12 | 0.07 |
| 7,500 | 60% | 141.00 | 379.00 | 100% | 0.20 | 0.14 |
| 10,000 | 67% | 14.00 | 15.10 | 100% | 0.31 | 0.20 |
| 12,500 | 73% | 21.30 | 24.00 | 100% | 0.56 | 0.20 |
| 15,000 | 93% | 11.30 | 16.80 | 100% | 0.73 | 0.20 |
| 17,500 | 80% | 22.00 | 20.00 | 100% | 1.28 | 0.40 |
| 20,500 | 87% | 10.70 | 23.90 | 100% | 0.34 | 0.38 |
| 22,500 | 87% | 11.30 | 13.20 | 100% | 0.39 | 0.32 |
| 25,000 | 93% | 23.60 | 27.10 | 100% | 0.54 | 0.44 |
| 27,500 | 93% | 14.40 | 20.40 | 100% | 0.88 | 0.43 |
| 30,000 | 93% | 11.80 | 14.30 | 100% | 0.68 | 0.35 |
| 40,000 | 100% | 23.40 | 23.70 | 100% | 1.67 | 1.06 |

Tables 7 and 8, below, show average DNA quantity measured across a series of egg concentrations in LAMP and TAQMAN experiments using DAX-8. This data shows that the DAX-8 protocol works using TAQMAN chemistry as well as LAMP, however TAQMAN generated a lower DNA quantity than LAMP.

TABLE 7
LAMP Average Data Quantity

| Eggs/ | | | | | | | | | | |
|---|---|---|---|---|---|---|---|---|---|---|
| 100 cc Soil | | 2,500 | 5,000 | 7,500 | 10,000 | 20,000 | 22,500 | 25,000 | 27,500 | R² |
| DNA | Avg | 0.26 | 0.64 | 1.62 | 0.41 | 1.38 | 3.72 | 1.32 | 5.17 | 0.552 |
| Quantity | StDev | N/A | 0.88 | 0.55 | 0.35 | 0.69 | 3.17 | N/A | N/A | |
| (pg) | | | | | | | | | | |

TABLE 8
TAQMAN Average Data Quantity

| Eggs/ | | | | | | | | | | |
|---|---|---|---|---|---|---|---|---|---|---|
| 100 cc Soil | | 2500 | 5,000 | 7,500 | 10,000 | 20,000 | 22,500 | 25,000 | 27,500 | R² |
| DNA | Avg | 5.38E−06 | 9.62E−05 | 1.15E−04 | 1.75E−04 | 1.33E−04 | 7.52E−05 | 1.54E−04 | 3.47E−04 | 0.404 |
| Quantity | StDev | N/A | 7.76E−05 | 1.00E−04 | 3.85E−05 | N/A | 9.14E−05 | N/A | 1.77E−04 | |
| (pg) | | | | | | | | | | |

### Example 2

### Data Quality and Sensitivity is Higher Using TAQMAN vs. LAMP.

The experiment described in Example 1 was repeated and subsequent data analysis revealed that compared to LAMP, TAQMAN has a lower limit of detection, a better R2 value for the titration curve, better quantification at lower egg count, and lower quantity output by about 1000 fold, which is reflected by the amplification Ct relative to the standard dilution. These results are described in the following tables.

Table 9 shows that although both marker technologies generate good titration data with Rapid Soil Pathogen Genotyping Version with DAX-8, TAQMAN has a better trendline R2 compared to LAMP. Table 10 further shows that TAQMAN had a better data return than LAMP.

TABLE 9

| Eggs/ | TAQMAN | | LAMP | |
|---|---|---|---|---|
| 100 cc | Avg | Stdev | Avg | Stdev |
| 0 | 2.63E−05 | N/A | No Amp | N/A |
| 100 | 4.61E−05 | 5.55E−05 | 1.94 | N/A |
| 250 | 8.43E−05 | 3.24E−05 | 1.19 | 0.86 |
| 500 | 2.10E−04 | 1.16E−04 | 0.51 | 0.59 |
| 750 | 1.69E−04 | 1.38E−04 | 1.69 | 1.27 |
| 1,000 | 1.53E−04 | N/A | No Amp | N/A |
| 2,500 | 6.56E−04 | 4.46E−04 | 1.78 | 0.72 |
| 5,000 | 1.12E−03 | N/A | 2.42 | N/A |
| 7,500 | 8.27E−04 | 4.01E−04 | 3.45 | 0.86 |
| 10,000 | 1.26E−03 | 1.33E−03 | 4.42 | 2.23 |
| 12,500 | 2.20E−03 | N/A | 4.27 | N/A |
| 15,000 | 3.09E−03 | 1.40E−03 | 5.22 | 2.02 |
| 17,500 | 2.04E−03 | 3.23E−04 | 2.78 | 0.68 |
| 22,500 | 4.53E−03 | 9.75E−04 | 5.5 | 1 |
| 25,000 | 3.72E−03 | 1.37E−03 | 5 | 0.8 |
| 27,500 | 5.40E−03 | 1.38E−03 | 5.4 | 1.2 |
| 30,000 | 5.62E−03 | 2.70E−03 | 5.7 | 0.9 |
| 40,000 | 8.33E−03 | 2.10E−03 | 8.3 | 1.4 |

| | | |
|---|---|---|
| R² | 0.9578 | 0.846 |

TABLE 10

| Eggs/ | TAQMAN | LAMP |
|---|---|---|
| 100 cc | % Data Return | % Data Return |
| 0 | 25% | 0% |
| 100 | 73% | 55% |
| 250 | 100% | 100% |
| 500 | 78% | 56% |
| 750 | 89% | 67% |
| 1,000 | 100% | 100% |
| 2,500 | 100% | 100% |
| 5,000 | 100% | 100% |
| 7,500 | 100% | 67% |
| 10,000 | 100% | 0% |
| 12,500 | 100% | 100% |
| 15,000 | 100% | 88% |
| 17,500 | 100% | 100% |
| 22,500 | 100% | 100% |
| 25,000 | 100% | 100% |
| 27,500 | 100% | 60% |
| 30,000 | 100% | 100% |
| 40,000 | 100% | 100% |

### Examples 3-4

### Additional Experiments Testing Amplification with TAQMAN

Two additional experiments were run to test TAQMAN amplification following DAX-8 treatment on soil samples spiked with SCN eggs. These experiments (described here as Examples 3 and 4) were run using 9 samples per egg count and were conducted as described in Example 1. Again, TAQMAN showed a good correlation between the number of SCN eggs spiked and DNA quantity/limit of detection. Here, the limit of detection was approximately 250 eggs/100 cc across both experiments. Table 11 depicts the results as % Data Return and DNA quantity obtained across the spiked samples for these two Examples.

TABLE 11

| | Example 3 | | | Example 4 | | |
|---|---|---|---|---|---|---|
| | | Average DNA | | | Average DNA | |
| Eggs/ | % Data | Quantity (pg) | | % Data | Quantity (pg) | |
| 100 cc | Return | Avg | Stdev | Return | Avg | Stdev |
| 0 | 22% | 2.06E−04 | 1.78E−04 | 22% | 2.80E−04 | 2.03E−04 |
| 50 | 67% | 6.40E−05 | 4.71E−05 | 22% | 1.39E−04 | 5.65E−06 |
| 100 | 22% | 8.23E−05 | 5.75E−05 | 33% | 2.11E−04 | 6.64E−05 |
| 250 | 89% | 5.85E−05 | 5.82E−05 | 78% | 2.08E−04 | 1.71E−04 |
| 500 | 78% | 4.70E−05 | 4.24E−05 | 67% | 2.44E−04 | 1.46E−04 |
| 750 | 100% | 1.05E−04 | 8.22E−05 | 89% | 3.71E−04 | 2.35E−04 |
| 1,000 | 100% | 1.08E−04 | 9.54E−05 | 100% | 4.48E−04 | 1.56E−04 |
| 1,250 | 100% | 1.37E−04 | 1.68E−04 | 78% | 3.66E−04 | 1.14E−04 |
| 1,500 | 89% | 7.41E−05 | 6.45E−05 | 78% | 3.32E−04 | 2.74E−04 |
| 1,750 | 89% | 1.73E−04 | 1.31E−04 | 89% | 3.58E−04 | 2.80E−04 |
| 2,000 | 100% | 1.68E−04 | 1.52E−04 | 89% | 4.01E−04 | 2.50E−04 |
| 3,000 | 100% | 3.08E−04 | 1.89E−04 | 100% | 1.15E−03 | 5.21E−04 |
| 4,000 | 100% | 2.97E−04 | 2.40E−04 | 100% | 1.34E−03 | 5.39E−04 |
| 5,000 | 100% | 5.88E−04 | 3.96E−04 | 89% | 1.31E−03 | 3.46E−04 |
| 7,500 | 100% | 7.62E−04 | 5.29E−04 | 100% | 1.35E−03 | 8.99E−04 |
| 10,000 | 100% | 1.04E−03 | 7.20E−04 | 100% | 2.00E−03 | 1.02E−03 |
| 12,500 | 100% | 1.06E−03 | 7.35E−04 | 100% | 3.25E−03 | 9.99E−04 |
| 15,000 | 100% | 1.43E−03 | 6.63E−04 | 100% | 3.78E−03 | 1.04E−03 |
| 17,500 | 100% | 2.42E−03 | 8.58E−04 | 100% | 4.19E−03 | 1.17E−03 |
| 20,000 | 100% | 2.15E−03 | 9.10E−04 | 100% | 5.41E−03 | 2.09E−03 |
| 22,250 | 100% | 3.11E−03 | 5.82E−04 | 100% | 2.91E−03 | 1.54E−03 |
| 25,000 | 100% | 3.98E−03 | 1.31E−03 | 100% | 4.44E−03 | 1.84E−03 |
| 27,500 | 100% | 4.01E−03 | 1.44E−03 | 100% | 4.73E−03 | 2.33E−03 |
| 30,000 | 100% | 3.74E−03 | 1.39E−03 | 100% | 6.10E−03 | 2.37E−03 |

| | |
|---|---|
| R² = 0.959 | R² = 0.919 |

### Example 5

### DAX-8 Outperforms XAD-4 with TAQMAN Technology

A series of soil samples spiked with SCN eggs were treated with XAD-4, another non-ionic resin capable of absorbing and releasing ionic species through hydrophobic and polar interactions, in the same way as described in Example 1 for DAX-8 (i.e., using the same ratio of lysate to resin). Good titration curves were only observed at 0.01× dilution and while XAD-4 had some success with LAMP amplification, it completely failed with TAQMAN. Table 12 shows the % Data Return measured for both LAMP and TAQMAN amplification experiments using XAD-4 and DAX-8 (data reproduced from Example 2). It is clear that although both resins effectively worked with LAMP amplification (although DAX-8 had a lower limit of detection), only DAX-8 worked with TAQMAN.

TABLE 12

| | DAX-8* | | XAD-4 | |
|---|---|---|---|---|
| Eggs/ | TAQMAN | LAMP | TAQMAN | LAMP |
| 100 cc | % Data Return | % Data Return | % Data Return | % Data Return |
| 0 | 25% | 0% | 25% | 0% |
| 100 | 73% | 55% | 18% | 27% |
| 250 | 100% | 100% | 13% | 38% |
| 500 | 78% | 56% | 0% | 44% |
| 750 | 89% | 67% | 11% | 67% |
| 1,000 | 100% | 100% | 0% | 100% |
| 2,500 | 100% | 100% | 0% | 100% |
| 5,000 | 100% | 100% | 0% | 100% |
| 7,500 | 100% | 67% | 33% | 100% |
| 10,000 | 100% | 0% | 0% | 100% |
| 12,500 | 100% | 100% | 0% | 100% |
| 15,000 | 100% | 88% | 25% | 100% |
| 17,500 | 100% | 100% | 0% | 100% |
| 20,000 | 0% | 0% | 0% | 100% |
| 22,500 | 100% | 100% | 33% | 100% |
| 25,000 | 100% | 100% | 17% | 100% |
| 27,500 | 100% | 60% | 40% | 100% |
| 30,000 | 100% | 100% | 0% | 100% |
| 40,000 | 100% | 100% | 20% | 100% |

*Data reproduced from Example 2.

### Example 6

### DAX-8 Out-Performs AlSO4 Treatment in LAMP Amplification

Aluminum sulfate is another chelator that binds positively charged molecules to pull out of solution. Table 13 shows average Ct values obtained from LAMP assays run on samples spiked with 0, 250 or 1,000 SCN eggs and treated with either DAX-8 or 2% AlSO₄. No amplification was observed in any sample using aluminum sulfate. Even non-treated samples showed some amplification late in the protocol, suggesting that AlSO₄ further interferes with amplification by binding DNA.

TABLE 13

| | qPCR Ct Value | | | | | |
|---|---|---|---|---|---|---|
| | Ave | StDev | Ave | StDev | Ave | StDev |
| DAX-8 | No Amp | No Amp | 81.89 | 5.24 | 70.27 | 2.69 |
| 2% AlSO₄ | No Amp | No Amp | No Amp | N/A | No Amp | N/A |
| No Treatment | No Amp | No Amp | 231.05 | 3.16 | 133.57 | N/A |

| | | | |
|---|---|---|---|
| Eggs/100 cc | 0 | 250 | 1,000 |

### Example 7

### Testing 11 More Resins Demonstrates Advantages of DAX-8

Eleven different resins were tested using the methods of Example 1 to determine their effectiveness at removing soil inhibitors during DNA extraction: Avicel (microcrystalline cellulose), Chitosan shrimp, Polyvinylpyrrolidone (PVP), XAD-7, XAD-16, Cellulose, Bentonite, Chitosan, Charcoal, Nano clay, XAD-2, and DAX-8. Each polymer was prepared in the same way as DAX-8 in Example 1 and added in a 1.5:1 ratio (resin: lysate). The absorbance of the extract (at 492 nm) was determined for each sample using a spectrophotometer before diluting to 0.1× or 0.01× and using in a standard LAMP or TAQMAN protocol. Table 14 describes the success of each reaction (using % data return) alongside the absorbance measured at 492 nm. Lower absorbance correlated strongly with a successful amplification reaction. While three polymers (XAD-7, XAD-16, DAX-8) resulted in generally successful LAMP reactions (particularly when the highest dilution (0.01×) was used), only one polymer (DAX-8) was successful with TAQMAN. Therefore, the ability of DAX-8 to remove humic acids and aid in DNA extraction is unique to DAX-8 and is not easily predicted based on its similarity to other polymers/resins/chelators.

TABLE 14

| | | | LAMP | | TAQMAN | |
|---|---|---|---|---|---|---|
| | | | (% Data | | (% Data | |
| | CAS- | Absorbance | Return) | | Return) | |
| Resin | Number | at 492 nm | 0.1X | 0.01X | 0.1X | 0.01X |
| Avicel | 9004-34-6 | 1.63 | DNS | DNS | DNS | DNS |
| Chitosan | 9012-76-4 | 1.28 | DNS | DNS | DNS | DNS |
| Shrimp | | | | | | |
| PVP | 9003-39-8 | 1.33 | DNS | DNS | DNS | DNS |
| XAD-7 | 37380-43-1 | 0.26 | 67% | 100% | DNS | DNS |
| XAD-16 | 104219-63-8 | 0.33 | DNS | 67% | DNS | DNS |
| DAX-8 | 11104-40-8 | 0.17 | 83% | 83% | 100% | 83% |
| Cellulose | 9004-34-6 | 1.69 | DNS | DNS | DNS | DNS |
| Bentonite | 1302-78-9 | 2.27 | DNS | DNS | DNS | DNS |
| Chitosan | 9012-76-4 | 1.43 | DNS | DNS | DNS | DNS |
| Charcoal | 7440-44-0 | 1.72 | DNS | DNS | DNS | DNS |
| Nano | 1302-78-9 | 0.88 | DNS | DNS | DNS | DNS |
| Clay | | | | | | |
| XAD-2 | 9060-05-3 | 1.80 | DNS | DNS | DNS | DNS |

*DNS: reaction did not succeed, no data return

Therefore, the ability to use a macroporous resin to isolate DNA in a soil mixture is highly dependent on the physical nature and properties of the resin.

### Example 8

### Effect of pH on Reaction Success

To test the effect of pH on the success of the amplification reaction, the procedure described in Example 1 was repeated with two different lysates. One was an older lysate, prepared about 60 days prior to the experiment and stored at 4° C., having a pH of around 10-11; the other was a newer lysate freshly prepared from a different soil source having a pH of 12 to 13. In each experiment, a 2:1 DAX: lysate ratio was used. The lysate with the lower pH (10 to 11) succeeded, while the other failed. Thus, the effectiveness of DAX appears to depend on the pH of the lysate mixture.

To determine the effect of the DAX/lysis solution on soil pH a variety of soils were tested for changes in pH after mixing with DAX/lysis buffer to form the “extraction reaction.” While the soils started with pH's ranging from 5 to 9.5, each one resulted in a final pH close to 13 (Table 15).

TABLE 15
Soil pH before and after extraction

| Soil | pH before | pH after |
|---|---|---|
| Sample No. | Extraction | Extraction |
| 501 | 5.55 | 9.52 |
| 583 | 5.82 | 12.46 |
| 511 | 6.05 | 12.64 |
| 441 | 8.81 | 12.88 |
| 468 | 9.07 | 12.82 |
| 798 | 9.12 | 13.01 |
| Extraction | | 13.03 |
| buffer | | |

### Example 9

### Effect of the DAX: Lysate Ratio on Titration Success

A series of experiments were run to test different DAX: lysate ratios and the results further confirmed that DAX success is tightly correlated to lysate pH. The DAX: lysate ratio in this example was the volumetric ratio of the extraction solution to the lysate (e.g., 0.5 ml DAX solution to 0.3 ml of lysate had about a 1.6:1 ratio). In this example, extraction solutions were prepared as described in Example 1 and had DAX: lysate ratios of 1:1, 2:1, and 1.5:1. In each case, the mass of DAX-8 used in each 3 cc volume of soil was calculated and presented in Table 16, below.

TABLE 16

| | | Mass of |
|---|---|---|
| | | Manufacture |
| DAX-8 to | | DAX-8 needed (g) |
| Lysate Ratio | [symbol] | per 3 cc soil |
| 1:1 | [symbol] | 0.27 |
| 1.5:1 | [symbol] | 0.40 |
| 2:1 | [symbol] | 0.53 |

[symbol]

indicates data missing or illegible when filed

Each lysate was further diluted 0.1× or 0.01× before amplification with PCR (TAQMAN) or LAMP. The results are described in Table 17, below, as % data return. When the DAX: lysate ratio was 1:1, LAMP succeeded at both 0.1× and 0.01× dilutions, but TAQMAN was only successful at the 0.01× dilution. It is also notable that at this dilution (0.01×) TAQMAN performed better than LAMP (achieving a higher percentage of data return). At the highest DAX: lysate ratio (2:1), only TAQMAN worked well at both dilutions; LAMP barely worked at 0.1× and failed at 0.01×. At the 1.5:1 ratio, both TAQMAN and LAMP were successful across both dilutions, demonstrating that this ratio (1.5:1) is ideal for preparing extracts for both LAMP and TAQMAN reactions.

TABLE 17

| | LAMP | | TAQMAN | |
|---|---|---|---|---|
| DAX:Lysate | (% Data return) | | (% Data Return) | |
| Ratio | 0.1× | 0.01× | 0.1× | 0.01× |
| 1:1 | 80 | 80 | 0 | 100 |
| 1.5:1 | 100 | 80 | 100 | 80 |
| 2:1 | 20 | 0 | 60 | 60 |

### Example 10

### Effect of DAX-8 Treatment and Humic Acid Concentration on Success of LAMP Reaction to Amplify Pathogen DNA

A series of tests were run to determine how residual humic acid and DAX-8 can interfere with LAMP amplification. A series of samples were spiked with increasing concentrations of humic acid (0 to 640 ppm) and either left untreated or treated with DAX-8. The time to amplification (Tp) using a LAMP protocol was recorded in each case. As the data depicted in Table 18 shows, when humic acid concentrations were low, the addition of DAX-8 appeared to block the successful reaction. Conversely, when humic acid levels were too high, amplification failed without the addition of DAX-8.

TABLE 18

| Humic | Time to Amplification | |
|---|---|---|
| Acid | (Min) | |
| (ppm) | No DAX-8 | DAX-8 |
| 0 | 10.14 | No Amp |
| 10 | 10.29 | No Amp |
| 20 | 10.44 | No Amp |
| 40 | 13.39 | 19.57 |
| 80 | No Amp | 18.12 |
| 160 | No Amp | 17.27 |
| 320 | No Amp | 14.42 |
| 640 | No Amp | 11.12 |

### Example 11

### A Field-Based Method of Extracting DNA from Soil Samples

In this example, a portable field-based method is described suitable for preparing soil samples in the field for nucleic acid amplification or other analysis.

A DAX-8 “extraction solution” was prepared as described in Example 1. 9 mL of lysis buffer comprising 100 mM NaOH, 2% Tween20 was added to the barrel of a 10 mL syringe attached to a 0.2 um filter. 6 mL of DAX-8 “extraction solution” was then added to the syringe. Approximately 3 cm3 (cc) of soil was added to the extraction buffer in the barrel of the syringe. This “extraction reaction” (soil and lysis buffer +DAX-8) was then mixed manually (shaking and inverting) until thoroughly mixed (approximately 5-30 seconds). A plunger was placed inside the barrel of the syringe and pressure applied to push the extraction reaction through the 0.2 μm in filter. A few drops (approximately 50-200 μL) of filtered lysate were collected (1X concentration). A 10 μL sample was removed from this filtered lysate for use in a LAMP reaction or TAQMAN reaction using the procedures described in Example 1. Thus, the filtered 1× lysates were used for LAMP and the TAQMAN reaction.

In a parallel experiment, an unfiltered lysate was prepared by repeating the same experiment but omitting the filtering step. In this experiment, the “extraction reaction” was allowed to settle by gravity. The top layer was then removed, diluted to 1×, 0.1× and 0.01× and used in TAQMAN and LAMP amplification reactions using standard techniques.

Table 19 depicts the data quantity determined using each amplification reaction at each dilution for the two methods (with or without filtering). As is apparent from the table, both LAMP and TAQMAN succeeded on filtered lysates at a high concentration (1×), but the unfiltered lysates required dilution to 0.1× or 0.01× to succeed with TAQMAN. Testing is undergoing for LAMP at all concentrations.

TABLE 19

| Method: | Syringe Filter | | Unfiltered | | | | | |
|---|---|---|---|---|---|---|---|---|
| Lysate | 1X | | 1X | | 0.1X | | 0.01X | |
| Dilution: | Avg | StDev | Avg | Stdev | Avg | Stdev | Avg | Stdev |
| LAMP | 18.87 | 0.37 | N/A | N/A | N/A | N/A | N/A | N/A |
| Tp (min) | | | | | | | | |
| TAQMAN | 33.95 | 0.41 | No | No | 29.07 | 0.15 | 29.58 | 0.14 |
| (Ct) | | | Amp | Amp | | | | |

### Example 12

### Prophetic Example to Optimize Lysate Preparation for In Field High Throughput Applications

The procedures described in Example 11 will be repeated to prepare a series of lysates having different dilutions (e.g., 1×, 0.1×, 0.01×) and/or level of filtration (e.g., syringe filtered or unfiltered). Each lysate will be tested using the TAQMAN and LAMP assays to optimize the procedure for high throughput systems for use in the field (where, for example, dilution and/or filtration may not be optimal). In this example, optimizing the procedure for an isothermal amplification technique (e.g., LAMP) will be a priority since equipping a high throughput machine with a thermocycler may not be practical (especially if it is meant to be used in the field). The results from this example will show that scaling the methods described herein to automated/mobile/high throughput systems may be possible without a diluting step.

In view of the above, it will be seen that the several objects of the invention are achieved and other advantageous results attained.

As various changes could be made in the above methods and apparatus without departing from the scope of the invention, it is intended that all matter contained in the above description and shown in the accompanying drawing[s] shall be interpreted as illustrative and not in a limiting sense.

What is claimed is:

1. A method for preparing an extract comprising polynucleotides, the method comprising:
mixing a sample comprising particulates and the polynucleotides with (1) a macroporous compound component comprising a macroporous compound, (2) a base, and (3) a solvent to form an extraction mixture, wherein the sample is obtained from a growing area; and
separating at least a portion of the polynucleotides from the extraction mixture to form the extract comprising polynucleotides and a first fraction comprising at least a portion of the macroporous compound component and particulates, wherein the concentration of particulates in the extraction mixture is greater than the concentration of particulates in the extract comprising polynucleotides.

2. The method of claim 1 wherein the ratio of the mass (g) of the macroporous compound component to the volume (cm³) of the particulates is at least about 1:1, at least about 1.2:1, at least about 1.4:1, or at least about 1.6:1.

3. The method of claim 1 or 2 wherein the ratio of the mass (g) of the macroporous compound component to the volume (cm³) of the particulates is from about 1:1 to about 2:1, from about 1.2:1 to about 2:1, from about 1.4: to about 2:1, from about 1.6:1 to about 2:1, from about 1:1 to about 1.8:1, from about 1.2:1 to about 1.8:1, from about 1.4: to about 1.8:1, from about 1.6:1 to about 1.8:1, from about 1:1 to about 1.6:1, from about 1.2:1 to about 1.6:1, or from about 1.4:1 to about 1.6:1.

4. The method of any one of claims 1 to 3 wherein the ratio of the mass (g) of the macroporous compound component to the mass (g) of the particulates is at least about 1:1, at least about 1.2:1, at least about 1.4:1, or at least about 1.6:1.

5. The method of any one of claims 1 to 4 wherein the ratio of the mass (g) of the macroporous compound component to the mass (g) of the particulates is from about 1:1 to about 2:1, from about 1.2:1 to about 2:1, from about 1.4: to about 2:1, from about 1.6:1 to about 2:1, from about 1:1 to about 1.8:1, from about 1.2:1 to about 1.8:1, from about 1.4: to about 1.8:1, from about 1.6:1 to about 1.8:1, from about 1:1 to about 1.6:1, from about 1.2:1 to about 1.6:1, or from about 1.4:1 to about 1.6:1.

6. The method of any one of claims 1 to 5 wherein the concentration of base in the extraction mixture is about 1 mM to about 500 mM, from about 50 mM to about 250 mM, or from about 75 mM to about 150 mM.

7. The method of claim 1 further comprising mixing a first portion of the solvent with the sample comprising particulates and the base to form a basic mixture; obtaining a lysate from the basic mixture; and mixing the macroporous compound component with a portion of the lysate to form the extraction mixture.

8. The method of claim 7 further comprising separating the basic mixture into a solid portion and a supernatant and obtaining the lysate from the supernatant.

9. The method of claim 8 comprising: separating the basic mixture into the solid portion and the supernatant using at least one separation techniques selected from the group consisting of filtration, membrane separation techniques, centrifugation, sedimentation, chelation, electromagnetic attraction, and combinations thereof

10. The method of any one of claims 7 to 9 wherein the basic mixture comprises about 1 mM to about 500 mM, from about 50 mM to about 250 mM, or from about 75 mM to about 150 mM of the base.

11. The method of any one of claims 7 to 10 wherein the concentration of the particulates in the basic mixture is about 20 wt. % to about 50 wt. %, from about 25 wt. % to about 45 wt. %, or from about 30 wt. % to about 35 wt. %.

12. The method of any one of claims 7 to 11 wherein the ratio of the volume of the sample comprising particulates (cm³) to the volume (mL) of the basic mixture is from about 1:1 to about 1:4, from about 1:2 to about 1:4, or about 1:3.

13. The method of any one of claims 7 to 12 wherein the volumetric ratio of the macroporous compound component to the portion of the lysate is about 1:1 to about 2:1, about 1.2:1 to about 1.8:1, or about 1.4:1 to about 1.6:1.

14. The method of any one of claims 1 to 13 wherein the macroporous compound component comprises a second portion of the solvent.

15. The method of claim 14 wherein the macroporous compound component has a macroporous compound content of from about 50 wt. % to about 99 wt. %, from about 60 wt. % to about 90 wt. % or from about 80 wt. % to about 90 wt. %.

16. The method of any one of claims 7 to 15 comprising forming an extraction mixture by mixing a macroporous compound component having a macroporous compound content from about 80 wt. % to about 90 wt. % in solvent with a portion of a lysate obtained from a basic mixture comprising about 75 mM to about 150 mM of a base and 25 wt. % to about 45 wt. % of particulates such that the volumetric ratio of the macroporous compound component to the lysate is about 1:1 to about 2:1, about 1.2:1 to about 1.8:1, or about 1.4:1 to about 1.6:1.

17. The method of any one of claims 1 to 16 wherein the pH of the extraction mixture is no greater than about 13, no greater than about 12.5, or no greater than about 12.

18. The method of any one of claims 1 to 17 wherein the pH of the extraction mixture is from about 10 to about 13, from about 10 to about 12.5, from about 10 to about 12, from about 11 to about 13, from about 11 to about 12.5, or from about 11 to about 12.

19. The method of any one of claims 1 to 18 wherein the sample further comprises humic acid.

20. The method of any one of claims 1 to 19 wherein the first fraction further comprises humic acid.

21. The method of claim 19 or 20 wherein the humic acid concentration in the extraction mixture is greater than the humic acid concentration in the extract.

22. The method of any one of claims 1 to 21 wherein the solvent comprises, consists essentially of, or consists of water.

23. The method of any one of claims 1 to 22 wherein the macroporous compound comprises a macroporous resin.

24. The method of any one of claims 1 to 23 wherein the macroporous compound comprises a polymethacrylic polymer and/or a polymethylmethacrylic polymer.

25. The method of any one of claims 1 to 24 wherein the macroporous compound comprises a macroporous resin having a mean pore diameter of from about 5 nm to about 100 nm, from about 5 nm to about 50 nm, from about 5 nm to about 30 nm, from about 10 nm to about 100 nm, from about 10 nm to about 50 nm, from about 10 nm to about 30 nm, from about 20 nm to about 100 nm, from about 20 nm to about 50 nm, from about 20 nm to about 30 nm, or from about 20 nm to about 25 nm.

26. The method any one of claims 1 to 25 wherein the macroporous compound comprises a macroporous resin having has a specific surface area of from about 50 m²/g to about 1000 m²/g, from about 50 m²/g to about 800 m²/g, from about 50 m²/g to about 500 m²/g, from about 50 m²/g to about 200 m²/g, from about 100 m²/g to about 1000 m²/g, from about 100 m²/g to about 800 m²/g, from about 100 m²/g to about 500 m²/g, from about 100 m²/g to about 300 m²/g, from about 100 m²/g to about 200 m²/g, or from about 125 m²/g to about 175 m²/g.

27. The method of any one of claims 1 to 26 wherein the macroporous compound comprises a hydrated macroporous resin.

28. The method of claim 27 wherein the hydrated macroporous resin has a density of from about 1 to about 1.2 g/mL, from about 1 to about 1.1 g/mL, or from about 1.05 to about 1.1 g/mL at 25° C.

29. The method of any one of claims 1 to 28 wherein the macroporous compound comprises DAX-8, XAD-7, XAD-16, and/or hydrates thereof.

30. The method of any one of claims 1 to 29 wherein the macroporous compound comprises DAX-8 and/or hydrate thereof.

31. The method of any one of claims 1 to 30 wherein the base comprises a strong base.

32. The method of any one of claims 1 to 31 wherein the base comprises NaOH.

33. The method of any one of claims 1 to 32 wherein the extraction mixture further comprises an emulsifying agent.

34. The method of claim 33 wherein the emulsifying agent comprises a nonionic surfactant.

35. The method of claim 33 or 34 wherein the emulsifying agent comprises a polysorbate.

36. The method of any one of claims 1 to 35 wherein the extract has an absorbance that is less than about 1, less than about 0.5, or less than about 0.2 when measured between about 400 and about 500 nm at 25° C.

37. The method of any one of claims 1 to 36 wherein the extract has an absorbance that is less than about 1, less than about 0.5, or less than about 0.2 when measured at 492 nm at 25° C.

38. The method of any one of claims 1 to 37 wherein separating the portion of the polynucleotides from the extraction mixture comprises at least one separation techniques selected from the group consisting of filtration, centrifugation, sedimentation, and combinations thereof.

39. The method of any one of claims 1 to 38 wherein the polynucleotides comprise at least one molecule selected from the group consisting of single-stranded DNA (ssDNA), single-stranded RNA (ssRNA), double-stranded DNA (dsDNA), double-stranded RNA (dsRNA), a RNA/DNA hybrid, and any combination thereof.

40. The method of any one of claims 1 to 39 wherein the concentration of particulates in the first fraction is greater than the concentration of particulates in the extract.

41. The method of any one of claims 1 to 40 wherein the particulates comprise soil particulates.

42. The method of any one of claims 1 to 41 wherein the sample is a soil core obtained from a growing area.

43. The method of any one of claims 1 to 42 wherein the particulates comprise one or more plant parts.

44. The method of any one of claims 1 to 43 wherein the particulates comprise a plant part selected from the group consisting of a leaf, stem, root, seed, and combinations thereof.

45. The method of any one of claims 1 to 44 wherein the sample obtained from a growing area is obtained from the rhizosphere of a plant.

46. The method of any one of claims 1 to 45 wherein the sample obtained from a growing area has a moisture content of no greater than about 50 wt. %, no greater than about 25 wt. %, no greater than about 10 wt. %, or no greater than about 5 wt. %.

47. A method of analyzing a polynucleotide, the method comprising:
preparing an extract comprising polynucleotides according to the method of any one of claims 1 to 46; and
detecting or identifying a polynucleotide in the extract.

48. The method of claim 47 further comprising amplifying a polynucleotide in the extract.

49. A method for amplifying a polynucleotide, the method comprising:
preparing an extract comprising polynucleotides according to the method of any one of claims 1 to 46; and
amplifying a polynucleotide in the extract.

50. The method of claim 48 or 49 wherein amplifying the polynucleotide comprises at least one amplification procedure selected from the group consisting of: a polymerase chain reaction (PCR), multiplex PCR, a reverse transcriptase reaction (RT), loop mediated isothermal amplification (LAMP), nucleic acid sequence based amplification (NASBA), self-sustained sequence replication (3SR), strand displacement amplification, helicase-dependent amplification, nicking enzyme amplification reaction, multiple displacement amplification, rolling circle amplification, ligase chain reaction, ramification amplification method, and combinations thereof.

51. The method of claim any one of claims 48 to 50 comprising amplifying the polynucleotide using a polymerase chain reaction (PCR) or loop mediated isothermal amplification (LAMP).

52. The method of any one of claims 48 to 51 further comprising monitoring the amplification of the polynucleotide.

53. The method of claim 52 wherein the monitoring comprises at least one procedure selected from the group consisting of end-time PCR with gel, quantitative real time (qRT) PCR, quantitative LAMP, quantitative nucleic acid sequence-based amplification (QT-NASBA), digital PCR, and combinations thereof.

54. The method of claim 52 or 53 wherein monitoring the amplification of the polynucleotide comprises using a fluorescent probe or nucleic acid marker.

55. The method of claim any one of claims 52 to 54 wherein monitoring the amplification of the polynucleotide comprises using SYBR-Green or a TAQMAN probe.

56. The method of any one of claims 47 to 55 wherein the polynucleotide comprises RNA and the method further comprises reverse transcribing the RNA into a cDNA transcript.

57. The method of claim 56 further comprising amplifying the cDNA transcript.

58. The method of claim 57 further comprising monitoring the amplification of the cDNA transcript.

59. The method of any one of claims 47 to 58 further comprising sequencing the polynucleotide.

60. The method of claim 59 wherein the sequencing of the polynucleotide comprises Sanger sequencing, pyrosequencing, sequence by synthesis, emulsion PCR, nanopore sequencing, and combinations thereof.

61. A method of detecting an organism or agent comprising a polynucleotide in a sample comprising particulates, the method comprising:
preparing an extract comprising polynucleotides from the sample according to the method of any one of claims 1 to 46;
analyzing a polynucleotide obtained from the extract; and
detecting the organism or agent in the sample based on the analysis of the polynucleotide.

62. The method of claim 61 wherein the organism or agent promotes, enhances, or increases the growth, survival, reproduction of at least one plant.

63. The method of claim 61 or 62 wherein the organism or agent comprises a plant pest or a plant pathogen.

64. The method of any one of claims 61 to 63 wherein the pathogen hinders the growth, survival, reproduction, or productivity of at least one plant.

65. The method of claim 63 or 64 wherein the pest or pathogen is selected from the group consisting of microorganisms, viruses, nematodes, fungi, bacteria, oomycetes, protozoa, phytoplasma, parasitic plants, insects, mites, gastropods, arthropods, moths, thrips, locusts, crickets, beetles, worms and combinations thereof.

66. The method of any one of claims 61 to 65 wherein the organism or agent does not noticeably alter the growth, survival, reproduction or productivity of a plant.

67. The method of any one of claims 61 to 66 wherein the organism or agent comprises a virus, a microbe, a fungus, an animal, a protist, a plant, a plant part or any combination thereof.

68. The method of any one of claims 61 to 67 wherein detecting the organism or agent further comprises phenotyping a plant for tolerance to a pathogen.

69. The method of any one of claims 61 to 68 wherein detecting the organism or agent further comprises determining whether a plant should be used in a breeding program.

70. The method of any one of claims 61 to 69 wherein detecting the organism or agent further comprises identifying an allele or quantitative trait locus (QTL) of a plant that is associated with disease tolerance.

71. The method of any one of claims 61 to 70 wherein analyzing the polynucleotide comprises any analysis method according to any one of claims 47 to 60.

72. A method for treating a plant or seed, the method comprising:
preparing an extract comprising polynucleotides according to the method of any one of claims 1 to 46;
analyzing a polynucleotide obtained from the extract; and
applying to the plant or locus thereof a treatment based on the analysis of the polynucleotide.

73. The method of claim 72 wherein the treatment comprises an agrochemical, an organism, a viral vector, a transfection agent.

74. The method of claim 72 or 73 comprising treating the plant or seed against a pathogen.

75. The method of any one of claims 72 to 74 wherein the treatment comprises a nematicide or fungicide.

76. The method of any one of claims 72 to 75 wherein the treatment comprises applying a beneficial organism to a plant.

77. The method of claim 76 wherein the beneficial organism is identified based on the analysis of the polynucleotide.

78. The method of any one of claims 72 to 77 wherein analyzing the polynucleotide comprises an analysis method according to claims 47 to 60.

79. The method of any one of claims 72 to 78 wherein the plant is a crop plant.

80. A mobile soil analysis system comprising:
a soil sample collection device;
at least one vessel sized and shaped to receive the soil sample and one or more analysis reagents;
a polynucleotide detector configured to receive at least a portion of the soil sample and one or more analysis reagents from the at least one vessel and identify and/or quantify polynucleotides in the soil sample, wherein the polynucleotide detector is configured to generate a polynucleotide signal;
a soil sample processor in communication with the polynucleotide detector and configured to analyze the soil sample at least in part based on the polynucleotide signal;
a tangible storage medium storing soil sample analysis instructions executable by the soil sample processor, wherein when the soil sample analysis instructions are executed by the soil sample processor, the polynucleotide signal is processed and the analytic data associated with the soil sample is stored on the tangible storage medium; and
a mobile platform supporting the at least one vessel and the polynucleotide detector.

81. The mobile soil analysis system of claim 80 wherein the soil sample collection device comprises an auger, diverter, bore, plug, or any combination thereof

82. The mobile soil analysis system of claim 80 or 81 wherein the polynucleotide detector comprises a polynucleotide sequencer and/or amplifier.

83. The mobile soil analysis system of any one of claims 80 to 82 wherein the at least one vessel comprises a plurality of vessels, each vessel sized and shaped to receive a respective soil sample and one or more analysis reagents.

84. The mobile soil analysis system of any one of claims 80 to 83 further comprising one or more agitators in fluid communication with the one or more vessels configured to mix the soil sample and one or more analysis reagents.

85. The mobile soil analysis system of any one of claims 80 to 84 further comprising one or more containers for receiving one or more analytical reagents, wherein the containers are in fluid communication with the one or more vessels.

86. The mobile soil analysis system of any one of claims 80 to 85 wherein the system is a high throughput system.

87. The mobile soil analysis system of any one of claims 80 to 86, wherein the mobile platform is structured and operable to traverse over or through a growing area.

88. The mobile soil analysis system of any one of claims 80 to 87 further comprising an extraction conduit for removing at least a portion of the soil sample and one or more analysis reagents from the at least one vessel and wherein the extraction conduit is in fluid communication with the polynucleotide detector.

89. The mobile soil analysis system of any one of claims 80 to 88 wherein the soil sample collection device further comprises one or more arms connected to the mobile platform.

90. The mobile soil analysis system of claim 89 wherein each arm is extendable and/or retractable with respect to the mobile platform to collect the soil sample.

91. The mobile soil analysis system of any one of claims 80 to 90 wherein the mobile platform is operable to traverse the surface of a growing area.

92. The mobile soil analysis system of any one of claims 80 to 91 wherein the mobile platform is operable to aerially traverse a growing area.

93. A mobile soil treatment system comprising:
the mobile soil analysis system of any one of claims 80 to 92;
a container for receiving an agrochemical formulation;
a dispenser for administering the agrochemical formulation to a soil collection, a growing area, a plant, a plant part, and/or locus thereof, wherein the dispenser is in fluid communication with the container.

94. The mobile soil treatment system of claim 93 wherein the dispenser is in communication with the soil sample processor and configured to dispense the agrochemical formulation based on the analytic data stored on the tangible storage medium.

95. The mobile soil treatment system of claim 93 or 94 wherein the dispenser includes an agrochemical formulation applicator and an applicator support constructed to support the agrochemical formulation applicator, the support supported by the mobile platform and movable with respect to the mobile platform to position the applicator for administering the agrochemical formulation.

96. The mobile soil treatment system of claim 95 wherein the applicator support comprises an arm connected to the mobile platform, the arm being extendable and/or retractable with respect to the mobile platform to position the applicator for administering the agrochemical formulation.

97. A method of selecting a plant for advancement in a breeding program comprising:
preparing an extract comprising polynucleotides according to the method of any one of claims 1 to 46;
analyzing a polynucleotide obtained from the extract to assign a genotype to a plant; and
selecting for advancement in a breeding pipeline a plant based on the results of the analysis of the polynucleotide.

98. The method of claim 97 wherein analyzing the polynucleotide comprises an analysis method according to claims 47 to 60.
